(19) United States
(12) Patent Application Publication
(10) Pub. No.: US 2026/0198510 A1
(43) Pub. Date: Jul. 16, 2026

(54) CROP TREATMENT

(71) Applicant: APS Biocontrol Limited, Dundee (GB)

(72) Inventors: Alison Blackwell, Dundee (GB); Kiri Mack, Dundee (GB)

(21) Appl. No.: 19/384,258

(22) Filed: Nov. 10, 2025

Related U.S. Application Data
(63) Continuation of application No. 17/426,186, filed on Jan. 28, 2020, now abandoned.

(30) Foreign Application Priority Data
Jan. 28, 2019 (GB) 1901156.8

(51) Int. Cl. A23B 7/155; A23B 2/729; A23B 9/28; C12N 7/00

(52) U.S. Cl. CPC A23B 7/155; A23B 2/729; A23B 9/28; C12N 7/00; C12N 2795/10121; C12N 2795/10122; C12N 2795/10131; C12N 2795/10221; C12N 2795/10222; C12N 2795/10231

(57) ABSTRACT

The presence of microorganisms in or on food can lead to, for example, disease, damage (loss of food), a shortening of shelf life, premature ripening, reduced yield, spoilage and/or rotting. Microbial contamination of food also represents a potential health risk, increasing the likelihood of food poisoning and the like. This disclosure provides methods, uses and compositions which may be exploited as a means to reduce or eliminate the presence of microorganisms on or in food and in turn, the various problems associated therewith. Provided is a method of decontaminating food or a food growing medium, substrate or compost of one or more microorganisms, said method comprising contacting the food and/or the food growing medium, substrate or compost with one or more phage.

### RELATED APPLICATIONS

This application is a continuation application of U.S. application Ser. No. 17/426,186, filed Jul. 28, 2021, which is a U.S. national stage application filed under 35 U.S.C. § 371, based on International Patent Application No. PCT/GB2020/050198, filed on Jan. 28, 2020, which claims the benefit of United Kingdom Patent Application No. GB1901156.8, filed on Jan. 28, 2019. The entire contents of each of the foregoing applications are incorporated herein by reference.

### FIELD OF THE INVENTION

Described herein are compositions which may be used to treat or prevent diseases which adversely affect mushroom crops. The disclosure also provides methods and uses which exploit the disclosed compositions.

### BACKGROUND OF THE INVENTION

The presence of certain microorganisms in or on crops can be associated with crop spoilage, rotting and/or disease. Fruit, vegetables (including fungi (such as mushrooms), root vegetables/tubers and edible (salad) leaves)) are all prone to microbial disease.

In some cases, microbial disease in crops leads to superficial or cosmetic damage which is unsightly and unappealing to the end consumer. Crops with cosmetic damage are often discarded. In other cases, microbial disease leads to more serious problems such as crop failure, rotting and spoilage.

The pathogens which result in crop spoilage are often bacterial and may be part of the crop's natural flora—or present in the soil, medium or substrate used during cultivation or farming.

Fruit, vegetables and/or fungi which are stored and/or placed in bags for sale, are particularly susceptible to microbial induced spoilage and/or rotting as the atmosphere, for example the humidity during storage and/or in a bag, can promote microbial growth. Furthermore, the harvesting and/or preparation process may cause damage to the food leaving them vulnerable to microbial infection. Additionally, crops may become contaminated as they are grown or cultured; this contamination may cause the crop to succumb to diseases. These diseases may spoil the crop and/or may reduce yield.

The UK's mushroom industry delivers sales values of £114M at the farm gate and £385M at consumer level. Retail sales of mushrooms are increasing annually by around 3%, ranking third in the UK's best-selling vegetables after potatoes and tomatoes. Despite this apparently thriving sector, the UK industry is facing challenges from high production costs and extremely strong European import competition. There is a significant opportunity for the UK mushroom industry but it requires innovative approaches to advance the sustainable intensification of production. Despite considerable investment and innovation in the industry, diseases can still have a significant impact on a grower's ability to achieve commercial quality and yields, whilst matching import prices.

Bacterial blotch of mushroom is a serious disease responsible for crop losses of up to 10% (this equates to approximately £20M industry losses per annum). This is one of the main mushroom diseases organisms and there is currently no effective control for this pathogen.

The provision of an effective treatment for this disease would provide an important boost to the UK industry. Disclosed herein is a novel bacteriophage based composition which can be used in the control of mushroom blotch.

### SUMMARY OF THE INVENTION

The presence of microorganisms in or on food can lead to, for example, disease, damage (loss of food), a shortening of shelf life, premature ripening, reduced yield, spoilage and/or rotting. Microbial contamination of food also represents a potential health risk, increasing the likelihood of food poisoning and the like. This disclosure provides methods, uses and compositions which may be exploited as a means to reduce or eliminate the presence of microorganisms on or in food and in turn, the various problems associated therewith.

In particular, the disclosure provides methods and compositions for use in the treatment, prevention and/or control of blotch pathogens. These compositions find particular application in commercial mushroom farming/cultivation. The compositions described herein represent a significant improvement over the prior art as there are currently no bacterial blotch products on the market or under development.

The disclosure is based on the finding that certain phage (and compositions comprising the same), may be exploited as a means to control disease, in particular blotch (mushroom blotch) type disease, in mushroom crops.

Mushroom blotch is one of the most serious pathogens of cultivated (or farmed) mushrooms.

It is primarily caused by Pseudomonas tolaasii, a Gram-negative, soil dwelling pathogenic bacteria. This pathogen releases a toxin (tolassin) on the mushroom surface, leading to brown lesions and pitting. Infection slows mushroom development, reducing yield: losses are commonly quantified as up to 10% with an economic impact of around £19m (production losses) per annum. Further loses may occur post-harvest and these may result in an unmarketable product, consumer rejections and potentially further loss of business. The disease is a global issue and the blotch pathogen is difficult to control, it being highly adapted to living in nutrient-poor soils, spreading easily through compost and casing (the term “casing” being a reference to the “casing layer”—a layer of material (moist or damp) placed on top of the substrate containing the mushroom mycelium); it is also easily spread by “splash dispersal” during irrigation or it is transferred mechanically by mushroom pickers or invertebrate (insect) pests.

The blotch pathogen is most often introduced into a mushroom growing facility (a “mushroom house”) via the casing material and/or air-borne dust. Indeed, even after treatment (for example by pasteurisation) the (bacterial) pathogen responsible for blotch remains present in most casing material.

Current practices aim to minimise the impact of the disease through reducing inoculum levels by irrigating the compost and casing with chlorinated water and altering environmental conditions by drying to reduce bacterial multiplication. However, the drying process is energy intensive and mushroom yield is decreased in lower humidity regimes (<90%). Moreover, these control methods are impractical in commercial mushroom production, which involves precise environmental control (including irrigation) to give multiple ‘flushes’ (or harvests) of mushrooms from pre-spawned compost (overlain with peat casing) from a single batch of mycelium. Control through broad-spectrum antibacterials is unsuitable, as many commensal bacteria within the casing are essential for mushroom initiation (e.g. Pseudomonas putida).

Bacterial blotch is characterised by the formation of lesions on mushroom tissue that are initially pale yellow progressing towards a golden yellow or rich chocolate brown colour. While the discoloration may be superficial (penetrating no more than 2 to 3 mm into the tissue), the underlying mushroom tissue may appear water soaked and grey or yellow-grey. Under favourable conditions, the spots (or blotches) may enlarge and coalesce; occasionally the blotches expand to cover the entire mushroom cap. The mushroom stem may also be affected. Blotches may appear when the mushrooms are in the early button stage, but can appear on mushrooms of any age. Blotches can appear on stored, harvested or refrigerated mushrooms or mushrooms that have been packed or over-wrapped with a watertight film.

During commercial mushroom farming processes, there are limited opportunities to apply products aimed at the control of disease. This is because these products usually require water for delivery and, as part of the mushroom growing process, irrigation is limited to the first few days of cultivation and in between mushroom flushes. As such, any product capable of targeting an economically important pathogen would be extremely attractive to commercial mushroom growers, in addition to the potential benefits of decreased wastage, energy savings, increased yield and the opportunity to extend their market share.

In a first aspect, there is provided a method of treating, controlling or preventing disease in a mushroom, said method comprising contacting the mushroom with one or more phage or compositions described herein. The methods of this disclosure may require “contact” (between the phage/composition and the mushroom) under conditions and for a time sufficient to de-contaminate the mushroom of any blotch causing bacteria and/or to inactivate, lyse or kill at least a portion of any blotch-causing bacteria present on the mushroom.

The method may further comprise the step of contacting the mushroom culture facility and substrate(s) with a phage or composition described herein.

A second aspect provides phage (for example one or more phage) or a composition comprising the same, for use in treating, controlling or preventing disease in a mushroom.

The methods, uses and compositions described herein may be applied (or further applied) to any one or more of:
the mushroom irrigation water;
the cultivation substrate or medium (the “compost”);
the casing layer;
the fruiting bodies (the developing mushrooms);
the harvested (or picked) crop; and/or stored crop.

It should be noted that when applying a composition of this invention to a substrate, medium or compost for use in mushroom farming, the substrate, medium or compost may be a pre- or post-spawned substrate, medium or compost. A “pre-spawned” substrate, medium or compost may not contain any mushroom spores and/or mycelium. Conversely, a “post-spawned” substrate, medium or compost may contain mushroom spores and/or mycelium.

The term “mushroom” may embrace any type or species of mushroom that is susceptible to a disease caused or contributed to by a pathogen that is inactivated, killed or lysed by a phage. For example, the term “mushroom” may embrace any mushroom species (type or strain) that is vulnerable to a disease caused or contributed to by Pseudomonas tolaasii (otherwise or occasionally (historically) known as: P. fluorescens biotype G, Pseudomonas tolaasii, Phytomonas tolassi, or Bacterium tolaasi). For example, the term “mushroom” may encompass Agaricus sp. including, for example, Agaricus bisporus.

A mushroom to be treated using a method or phage described herein, may be part of a crop. A “mushroom crop” may comprise mushrooms of any type and may relate to any private or commercial mushroom cultivation process. For example, a mushroom crop may be a small scale (for example private/domestic) crop or a larger commercial type crop.

Mushroom blotch (also known as bacterial blotch, brown blotch and/or bacterial spot) may be caused by Pseudomonas tolaasii (P. fluorescens biotype G). As such, Pseudomonas tolaasii may be embraced within the term “blotch-causing bacteria”. For convenience, all blotch-type diseases affecting mushrooms, shall be referred to by use of the term “bacterial blotch”.

As stated, bacterial blotch is a serious disease affecting mushrooms and the methods, uses and compositions described herein may be used to treat, control or prevent the pathogens and/or bacterial blotch disease in mushrooms. Thus, in one embodiment, a method of this disclosure is a method of treating or preventing mushroom bacterial blotch, said method comprising contacting mushrooms with one or more of the phage or compositions described herein.

A further embodiment provides phage or a composition comprising the same (for example a composition comprising one or more phage) for use in treating, controlling or preventing mushroom bacterial blotch.

For the avoidance of doubt, it should be noted that throughout this specification, the terms “comprise” and/or “comprising” are used to denote that aspects and/or embodiments of the invention “comprise” the noted features and as such, may also include other features. However, in the context of this invention, the terms “comprise” and “comprising” encompass embodiments in which the invention “consists essentially of” the relevant features or “consists of” the relevant features.

It should be understood that the methods, composition and uses described herein may not only be applied to mushroom crops as they are growing, but to spores before they are seeded into a substrate and/or to mushrooms that are being processed (harvested and cleaned) and/or packed for storage.

Thus, as explained in more detail below, the methods described herein may exploit one or more phage types, the selected phage each being specific for those microorganisms (bacteria) which cause (or contribute to) the blotch type diseases described herein.

Without wishing to be bound by theory, phage for use in the various aspects and embodiments described herein may be described as either lytic or lysogenic. Lytic phage for use may include (but are not limited to) those that kill, inactivate and/or inhibit their target bacteria by first injecting their viral DNA or RNA and then replicating to induce microbial or bacterial cell death by, for example, lysis. Useful lysogenic phage may incorporate their nucleic acid (DNA or RNA) into the host and at some later stage (perhaps in response to a stress stimulus, for example UV light exposure) will replicate and cause bacterial cell death. In accordance with this invention, the decontamination (by lytic or lysogenic means) of bacteria present on or in mushrooms (as contaminants or otherwise) may result in the destruction, inactivation and/or killing of one or more bacteria and/or the inactivation or killing of a proportion (for example about 0.001% to about 1%, about 10%, about 20%, about 30%, about 40%, about 50%, about 60%, about 65%, about 70%, about 75%, about 80%, about 85%, about 90%, about 95%, about 96%, about 97%, about 98%, about 99% or about 100%) of a population of the blotch-causing bacteria present on or in the mushroom (or the environment/culture media).

Phage for use according to this disclosure may be obtained (or are obtainable) by methods which identify phage with an ability to lyse (kill) the target bacteria—in this case, those bacteria that are responsible for mushroom blotch diseases. Bacteria which cause or contribute to mushroom blotch disease may be isolated from samples of soil/casing (or some other culture substrate), mushrooms themselves (the fruiting bodies and/or mycelium), the environment and/or the irrigation system (water used in the growing of mushrooms and/or water used to wash, for example, harvested and stored mushrooms). Bacteria associated with mushroom blotch may also be isolated from mushroom samples that symptoms of the same (described above). Thus methods which identify phage suitable for use according to this disclosure may comprise, contacting a blotch-causing bacteria (including any of the specific bacteria described herein) with a phage and/or phage composition and determining whether or not the phage and/or phage composition is able to lyse, kill or destroy the bacteria. In such methods, the phage or phage composition may be referred to as a “test phage/composition”. The bacteria may have been isolated from a soil, compost, casing or mushroom sample. Phage which exhibit an ability to lyse (or are lysogenic) or kill/destroy bacteria which cause or contribute to mushroom blotch, may be used in the methods, uses and compositions of this invention.

One of skill will appreciate that variety and geographical origin of a particular mushroom may dictate its microbial flora and/or the type of microorganism most likely to be associated with blotch-type diseases. For example, mushrooms from one geographical location may be susceptible or blotch diseases caused or contributed to by one or more bacteria, and the same mushroom from another geographical location may be susceptible to blotch disease caused by one or more different bacteria. By obtaining samples of blotch affected mushroom from around the world, from different varieties (of the same mushroom and/or from mushrooms exhibiting symptoms of different types blotch disease, it should be possible to provide phage (and compositions comprising the same) which are not only effective against mushroom blotch bacteria from a range of geographic areas, but also useful in treating or preventing blotch in different varieties of the same mushroom and different types of blotch disease. A phage or phage composition may be modified and/or supplemented with additional phage of the same or different families to target other microorganisms.

The disclosure may exploit single or multiple phage types, each phage exhibiting a specificity and/or affinity for a single or multiple different bacteria. For example, where a particular mushroom exhibits a particular microbial/bacterial flora or is likely to be affected by (a) specific microbial contaminant(s) (for example a specific blotch causing bacteria), then any composition for use may be formulated so as to contain phage types which are specific to those microorganisms/bacteria. In other words, the phage for use may comprise one or more different phage types each with specificity or affinity for one or more of the relevant bacteria.

Phage for use may be derived from one or more taxonomic groups. Where the invention concerns the use of bacteriophage, tailed-phage of the order Caudovirales may be used. For example, bacteriophage belonging to the families Myoviridae, Podoviridae and/or Siphoviridae may be used. One of skill will appreciate that any selected Myoviridae, Podoviridae and/or Siphoviridae type phage may have an affinity for one or more of the species/strains of bacteria that cause or contribute to mushroom-blotch (such as, for example, Pseudomonas tolaasii).

Thus, the disclosure provides a method of decontaminating mushrooms of one or more blotch-causing bacteria or for treating and/or preventing mushroom blotch, said method comprising contacting the food with one or more Myoviridae, Podoviridae and/or Siphoviridae bacteriophage. The bacteria against which a method of this embodiment may be used, may include Pseudomonas tolaasii.

The disclosure further provides compositions for use in (i) the decontamination of mushrooms of one or more blotch causing bacteria or (ii) treating and/or preventing mushroom blotch, said composition comprising one or more bacteriophage selected from the group consisting of Myoviridae, Podoviridae and/or Siphoviridae bacteriophage. Again, the bacteria against which the composition of this embodiment may be used, may include Pseudomonas tolaasii.

The disclosure may exploit one or more bacteriophage selected from the group consisting of:
(i) APS MP1 (deposited as NCIMB 42864);
(ii) APS MP2 (deposited as NCIMB 42866);
(iii) APS MP3 (deposited as NCIMB 42868);
(iv) APS MP4 (deposited as NCIMB 43014);
(v) APS MP5 (deposited as NCIMB 43016);
(vi) APS MP6 (deposited as NCIMB 43018);
(vii) APS MP7 (deposited as NCIMB 43467);
(viii) APS MP8 (deposited as NCIMB 43469);
(ix) APS MP9 (deposited as NCIMB 43471);
(x) APS MP10 (deposited as NCIMB 43473); and
(xi) APS MP11 (deposited as NCIMB 43575).

For example, a composition of this invention may comprise one, two, three, four, five, six, seven, eight, nine, ten or all eleven of the bacteriophage listed as (i), (ii), (iii), (iv), (v), (vi), (vii), (viii), (ix), (x) and (xi) above.

For example a composition of this disclosure may exploit one or more of the bacteriophage listed in Table 1 below.

TABLE 1

| | | | NCIMB | Bacterial Host | | NCIMB | |
|---|---|---|---|---|---|---|---|
| Laboratory Phage | | NCIMB | Deposit | Laboratory | NCIMB | Deposit | Patent |
| Designation | | Designation | Number | Designation | Designation | Number | reference |
| P1 | MW141215_1.02 | Pseudomonas | 42864 | MW141215_1.02 | Pseudomonas | 42898 | APS |
| | P1 | tolaasii | | | tolaasii | | MP1 |
| | | APS MP1 | | | APS MP1 | | |
| | | Phage | | | | | |
| P2 | MW310715_1.08 | Pseudomonas | 42866 | MW310715_1.08 | Pseudomonas | 42899 | APS |
| | P2 | tolaasii | | | tolaasii | | MP2 |
| | | APS MP2 | | | APS MP2 | | |
| | | phage | | | | | |
| P51 | AS150615_1.06 | Pseudomonas | 42868 | AS150615_1.06 | Pseudomonas | 42900 | APS |
| | P51 | tolaasii | | | tolaasii | | MP3 |
| | | APS MP3 | | | APS MP3 | | |
| | | Phage | | | | | |
| 1.20P3 | TW012216_1.20.3 | Pseudomonas | 43014 | TW012216_1.20.3 | Pseudomonas | 43015 | APS |
| | | tolaasii | | | tolaasii | | MP4 |
| | | APS MP4 | | | APS MP4 | | |
| | | Phage | | | | | |
| 1_15 P3 | MW141215_1.15 | Pseudomonas | 43016 | MW141215_1.15 | Pseudomonas | 43017 | APS |
| | P3 | tolaasii | | | tolaasii | | MPS |
| | | APS MP5 | | | APS MP5 | | |
| | | Phage | | | | | |
| E1P1 | MF171017_4.5 | Pseudomonas | 43018 | MF171017_4.5 | Pseudomonas | 43019 | APS |
| | | tolaasii | | | tolaasii | | MP6 |
| | E1P1 | APS MP6 | | | APS MP6 | | |
| | | Phage | | | | | |
| | MF180915_1.04 | Pseudomonas | 43467 | MF180915_1.04 | Pseudomonas | 43468 | APS |
| | | tolaasii | | P3 | tolaasii | | MP7 |
| | | APS MP07 | | | APS MP7 | | |
| | | | | | Phage | | |
| | 241017_4.11 | Pseudomonas | 43469 | 241017_4.11 | Pseudomonas | 43470 | APS |
| | | tolaasii | | P2 | tolaasii | | MP8 |
| | | APS MP08 | | | APS MP8 | | |
| | | | | | Phage | | |
| | MF060717_1.24 | Pseudomonas | 43471 | MF060717_1.24 | Pseudomonas | 43472 | APS |
| | | tolaasii | | P2 | tolaasii | | MP9 |
| | | APS MP09 | | | APS MP9 | | |
| | | | | | Phage | | |
| | MF051017_1.29 | Pseudomonas | 43473 | MF051017_1.29 | Pseudomonas | 43474 | APS |
| | | tolaasii | | P1 | tolaasii | | MP10 |
| | | APS MP10 | | | APS MP10 | | |
| | | | | | Phage | | |
| | MF310818_1.3 | Pseudomonas | 43475 | MF310818_1.3 | Pseudomonas | 43476 | APS |
| | | tolaasii | | P3 | tolaasii | | MP11 |
| | | APS MP11 | | | APS MP11 | | |
| | | | | | Phage | | |

Insofar as the disclosure relates to a method, use and/or composition, it may exploit at least two or at least three bacteriophage selected from the group consisting of bacteriophage (i) to (xi) listed above and in Table 1.

For example, the various uses, methods and compositions of the disclosure may exploit a “core phage composition” comprising (or “consisting essentially” or “consisting” of), bacteriophage APS MP1 (MW141215_1.02 P1: deposited as NCIMB 42864). This core phage composition may optionally comprise one or more of the other bacteriophage described herein.

For example, a use, method and/or composition comprising APS MP1 may further comprise one or more bacteriophage selected from the group consisting of:
bacteriophage APS MP2; (deposited as NCIMB 42866)
bacteriophage APS MP3; (deposited as NCIMB 42868)
bacteriophage APS MP4; (deposited as NCIMB 43014)
bacteriophage APS MP5; (deposited as NCIMB 43016)
bacteriophage APS MP6. (deposited as NCIMB 43018)
bacteriophage APS MP7 (deposited as NCIMB 43467);
bacteriophage APS MP8 (deposited as NCIMB 43469);
bacteriophage APS MP9 (deposited as NCIMB 43471);
bacteriophage APS MP10 (deposited as NCIMB 43473); and
bacteriophage APS MP11 (deposited as NCIMB 43575).

As such, the present disclosure provides compositions comprising the following bacteriophage:
APS MP1; or
APS MP2; or
APS MP3; or
APS MP4 or
APS MP5; or
APS MP6; or
APS MP7; or
APS MP8; or
APS MP9; or
APS MP10; or
APS MP11.

The present disclosure provides compositions comprising the following bacteriophage combinations (two bacteriophage types per composition):

$$APS MP 1 + APS MP 2 ; or$$

$$APS MP 1 + APS MP 3 ; or$$

$$APS MP 1 + APS MP 4 ; or$$

$$APS MP 1 + APS MP 5 ; or$$

$$APS MP 1 + APS MP 6 ; or$$

$$APS MP 1 + APS MP 7 ; or$$

$$APS MP 1 + APS MP 8 ; or$$

$$APS MP 1 + APS MP 9 ; or$$

$$APS MP 1 + APS MP 10 ; or$$

$$APS MP 1 + APS MP 11 ; or$$

$$APS MP 2 + APS MP 3 ; or$$

$$APS MP 2 + APS MP 4 ; or$$

$$APS MP 2 + APS MP 5 ; or$$

$$APS MP 2 + APS MP 6 ; or$$

$$APS MP 2 + APS MP 7 ; or$$

$$APS MP 2 + APS MP 8 ; or$$

$$APS MP 2 + APS MP 9 ; or$$

$$APS MP 2 + APS MP 10 ; or$$

$$APS MP 2 + APS MP 11 ; or$$

$$APS MP 3 + APS MP 4 ; or$$

$$APS MP 3 + APS MP 5 ; or$$

$$APS MP 3 + APS MP 6 ; or$$

$$APS MP 3 + APS MP 7 ; or$$

$$APS MP 3 + APS MP 8 ; or$$

$$APS MP 3 + APS MP 9 ; or$$

$$APS MP 3 + APS MP 10 ; or$$

$$APS MP 3 + APS MP 11 ; or$$

$$APS MP 4 + APS MP 5 ; or$$

$$APS MP 4 + APS MP 6 ; or$$

$$APS MP 4 + APS MP 7 ; or$$

$$APS MP 4 + APS MP 8 ; or$$

$$APS MP 4 + APS MP 9 ; or$$

$$APS MP 4 + APS MP 10 ; or$$

$$APS MP 4 + APS MP 11 ; or$$

$$APS MP 5 + APS MP 6 ; or$$

$$APS MP 5 + APS MP 7 ; or$$

$$APS MP 5 + APS MP 8 ; or$$

$$APS MP 5 + APS MP 9 ; or$$

$$APS MP 5 + APS MP 10 ; or$$

$$APS MP 5 + APS MP 11 ; or$$

$$APS MP 6 + APS MP 7 ; or$$

$$APS MP 6 + APS MP 8 ; or$$

$$APS MP 6 + APS MP 9 ; or$$

$$APS MP 6 + APS MP 10 ; or$$

$$APS MP 6 + APS MP 11 ; or$$

$$APS MP 7 + APS MP 8 ; or$$

$$APS MP 7 + APS MP 9 ; or$$

$$APS MP 7 + APS MP 10 ; or$$

$$APS MP 7 + APS MP 11 ; or$$

$$APS MP 8 + APS MP 9 ; or$$

$$APS MP 8 + APS MP 10 ; or$$

$$APS MP 8 + APS MP 11 ; or$$

$$APS MP 9 + APS MP 10 ; or$$

$$APS MP 9 + APS MP 11 ; or$$

$$APS MP 10 + APS MP 11.$$

The present disclosure provides compositions comprising the following bacteriophage combinations (three bacteriophage types per composition):

$$APS MP 1 + APS MP 2 + APS MP 3 ; or$$

$$APS MP 1 + APS MP 2 + APS MP 4 ; or$$

$$APS MP 1 + APS MP 2 + APS MP 5 ; or$$

$$APS MP 1 + APS MP 2 + APS MP 6 ; or$$

$$APS MP 1 + APS MP 2 + APS MP 7 ; or$$

$$APS MP 1 + APS MP 2 + APS MP 8 ; or$$

$$APS MP 1 + APS MP 2 + APS MP 9 ; or$$

$$APS MP 1 + APS MP 2 + APS MP 10 ; or$$

$$APS MP 1 + APS MP 2 + APS MP 11 ; or$$

$$APS MP 1 + APS MP 3 + APS MP 4 ; or$$

$$APS MP 1 + APS MP 3 + APS MP 5 ; or$$

$$APS MP 1 + APS MP 3 + APS MP 6 ; or$$

$$APS MP 1 + APS MP 3 + APS MP 7 ; or$$

$$APS MP 1 + APS MP 3 + APS MP 8 ; or$$

$$APS MP 1 + APS MP 3 + APS MP 9 ; or$$

$$APS MP 1 + APS MP 3 + APS MP 10 ; or$$

$$APS MP 1 + APS MP 3 + APS MP 11 ; or$$

$$APS MP 1 + APS MP 4 + APS MP 5 ; or$$

$$APS MP 1 + APS MP 4 + APS MP 6 ; or$$

$$APS MP 1 + APS MP 4 + APS MP 7 ; or$$

$$APS MP 1 + APS MP 4 + APS MP 8 ; or$$

$$APS MP 1 + APS MP 4 + APS MP 9 ; or$$

$$APS MP 1 + APS MP 4 + APS MP 10 ; or$$

$$APS MP 1 + APS MP 4 + APS MP 11 ; or$$

$$APS MP 1 + APS MP 5 + APS MP 6 ; or$$

$$APS MP 1 + APS MP 5 + APS MP 7 ; or$$

$$APS MP 1 + APS MP 5 + APS MP 8 ; or$$

$$APS MP 1 + APS MP 5 + APS MP 9 ; or$$

$$APS MP 1 + APS MP 5 + APS MP 10 ; or$$

$$APS MP 1 + APS MP 5 + APS MP 11 ; or$$

$$APS MP 1 + APS MP 6 + APS MP 7 ; or$$

$$APS MP 1 + APS MP 6 + APS MP 8 ; or$$

$$APS MP 1 + APS MP 6 + APS MP 9 ; or$$

$$APS MP 1 + APS MP 6 + APS MP 10 ; or$$

$$APS MP 1 + APS MP 6 + APS MP 7 ; or$$

$$APS MP 1 + APS MP 7 + APS MP 8 ; or$$

$$APS MP 1 + APS MP 7 + APS MP 9 ; or$$

$$APS MP 1 + APS MP 7 + APS MP 10 ; or$$

$$APS MP 1 + APS MP 7 + APS MP 1 ; or$$

$$APS MP 1 + APS MP 8 + APS MP 9 ; or$$

$$APS MP 1 + APS MP 8 + APS MP 10 ; or$$

$$APS MP 1 + APS MP 8 + APS MP 11 ; or$$

$$APS MP 1 + APS MP 9 + APS MP 10 ; or$$

$$APS MP 1 + APS MP 10 + APS MP 11 ; or$$

$$APS MP 2 + APS MP 3 + APS MP 4 ; or$$

$$APS MP 2 + APS MP 3 + APS MP 5 ; or$$

$$APS MP 2 + APS MP 3 + APS MP 6 ; or$$

$$APS MP 2 + APS MP 3 + APS MP 7 ; or$$

$$APS MP 2 + APS MP 3 + APS MP 8 ; or$$

$$APS MP 2 + APS MP 3 + APS MP 9 ; or$$

$$APS MP 2 + APS MP 3 + APS MP 10 ; or$$

$$APS MP 2 + APS MP 3 + APS MP 11 ; or$$

$$APS MP 2 + APS MP 4 + APS MP 5 ; or$$

$$APS MP 2 + APS MP 4 + APS MP 6 ; or$$

$$APS MP 2 + APS MP 4 + APS MP 7 ; or$$

$$APS MP 2 + APS MP 4 + APS MP 8 ; or$$

$$APS MP 2 + APS MP 4 + APS MP 9 ; or$$

$$APS MP 2 + APS MP 4 + APS MP 10 ; or$$

$$APS MP 2 + APS MP 4 + APS MP 11 ; or$$

$$APS MP 2 + APS MP 5 + APS MP 6 ; or$$

$$APS MP 2 + APS MP 5 + APS MP 7 ; or$$

$$APS MP 2 + APS MP 5 + APS MP 8 ; or$$

$$APS MP 2 + APS MP 5 + APS MP 9 ; or$$

$$APS MP 2 + APS MP 5 + APS MP 10 ; or$$

$$APS MP 2 + APS MP 5 + APS MP 11 ; or$$

$$APS MP 2 + APS MP 6 + APS MP 7 ; or$$

$$APS MP 2 + APS MP 6 + APS MP 8 ; or$$

$$APS MP 2 + APS MP 6 + APS MP 9 ; or$$

$$APS MP 2 + APS MP 6 + APS MP 10 ; or$$

$$APS MP 2 + APS MP 6 + APS MP 11 ; or$$

$$APS MP 2 + APS MP 7 + APS MP 8 ; or$$

$$APS MP 2 + APS MP 7 + APS MP 9 ; or$$

$$APS MP 2 + APS MP 7 + APS MP 10 ; or$$

$$APS MP 2 + APS MP 7 + APS MP 11 ; or$$

$$APS MP 2 + APS MP 8 + APS MP 9 ; or$$

$$APS MP 2 + APS MP 8 + APS MP 10 ; or$$

$$APS MP 2 + APS MP 8 + APS MP 11 ; or$$

$$APS MP 2 + APS MP 9 + APS MP 10 ; or$$

$$APS MP 2 + APS MP 9 + APS MP 11 ; or$$

$$APS MP 2 + APS MP 10 + APS MP 11 ; or$$

$$APS MP 3 + APS MP 4 + APS MP 5 ; or$$

$$APS MP 3 + APS MP 4 + APS MP 6 ; or$$

$$APS MP 3 + APS MP 4 + APS MP 7 ; or$$

$$APS MP 3 + APS MP 4 + APS MP 8 ; or$$

$$APS MP 3 + APS MP 4 + APS MP 9 ; or$$

$$APS MP 3 + APS MP 4 + APS MP 10 ; or$$

$$APS MP 3 + APS MP 4 + APS MP 11 ; or$$

$$APS MP 3 + APS MP 5 + APS MP 6 ; or$$

$$APS MP 3 + APS MP 5 + APS MP 7 ; or$$

$$APS MP 3 + APS MP 5 + APS MP 8 ; or$$

$$APS MP 3 + APS MP 5 + APS MP 9 ; or$$

$$APS MP 3 + APS MP 5 + APS MP 10 ; or$$

$$APS MP 3 + APS MP 5 + APS MP 11 ; or$$

$$APS MP 3 + APS MP 6 + APS MP 7 ; or$$

$$APS MP 3 + APS MP 6 + APS MP 8 ; or$$

$$APS MP 3 + APS MP 6 + APS MP 9 ; or$$

$$APS MP 3 + APS MP 6 + APS MP 10 ; or$$

$$APS MP 3 + APS MP 6 + APS MP 11 ; or$$

$$APS MP 3 + APS MP 7 + APS MP 8 ; or$$

$$APS MP 3 + APS MP 7 + APS MP 9 ; or$$

$$APS MP 3 + APS MP 7 + APS MP 10 ; or$$

$$APS MP 3 + APS MP 7 + APS MP 11 ; or$$

$$APS MP 3 + APS MP 8 + APS MP 9 ; or$$

$$APS MP 3 + APS MP 8 + APS MP 10 ; or$$

$$APS MP 3 + APS MP 8 + APS MP 11 ; or$$

$$APS MP 3 + APS MP 9 + APS MP 10 ; or$$

$$APS MP 3 + APS MP 9 + APS MP 11 ; or$$

$$APS MP 3 + APS MP 10 + APS MP 11 ; or$$

$$APS MP 4 + APS MP 5 + APS MP 6 ; or$$

$$APS MP 4 + APS MP 5 + APS MP 7 ; or$$

$$APS MP 4 + APS MP 5 + APS MP 8 ; or$$

$$APS MP 4 + APS MP 5 + APS MP 9 ; or$$

$$APS MP 4 + APS MP 5 + APS MP 10 ; or$$

$$APS MP 4 + APS MP 5 + APS MP 11 ; or$$

$$APS MP 4 + APS MP 6 + APS MP 7 ; or$$

$$APS MP 4 + APS MP 6 + APS MP 8 ; or$$

$$APS MP 4 + APS MP 6 + APS MP 9 ; or$$

$$APS MP 4 + APS MP 6 + APS MP 10 ; or$$

$$APS MP 4 + APS MP 6 + APS MP 11 ; or$$

$$APS MP 4 + APS MP 7 + APS MP 8 ; or$$

$$APS MP 4 + APS MP 7 + APS MP 9 ; or$$

$$APS MP 4 + APS MP 7 + APS MP 10 ; or$$

$$APS MP 4 + APS MP 7 + APS MP 11 ; or$$

$$APS MP 4 + APS MP 8 + APS MP 9 ; or$$

$$APS MP 4 + APS MP 8 + APS MP 10 ; or$$

$$APS MP 4 + APS MP 8 + APS MP 11 ; or$$

$$APS MP 4 + APS MP 9 + APS MP 10 ; or$$

$$APS MP 4 + APS MP 9 + APS MP 11 ; or$$

$$APS MP 4 + APS MP 10 + APS MP 11 ; or$$

$$APS MP 5 + APS MP 6 + APS MP 7 ; or$$

$$APS MP 5 + APS MP 6 + APS MP 8 ; or$$

$$APS MP 5 + APS MP 6 + APS MP 9 ; or$$

$$APS MP 5 + APS MP 6 + APS MP 10 ; or$$

$$APS MP 5 + APS MP 6 + APS MP 11 ; or$$

$$APS MP 5 + APS MP 7 + APS MP 8 ; or$$

$$APS MP 5 + APS MP 7 + APS MP 9 ; or$$

$$APS MP 5 + APS MP 7 + APS MP 10 ; or$$

$$APS MP 5 + APS MP 7 + APS MP 11 ; or$$

$$APS MP 5 + APS MP 8 + APS MP 9 ; or$$

$$APS MP 5 + APS MP 8 + APS MP 10 ; or$$

$$APS MP 5 + APS MP 8 + APS MP 11 ; or$$

$$APS MP 5 + APS MP 9 + APS MP 10 ; or$$

$$APS MP 5 + APS MP 9 + APS MP 11 ; or$$

$$APS MP 5 + APS MP 10 + APS MP 11 ; or$$

$$APS MP 6 + APS MP 7 + APS MP 8 ; or$$

$$APS MP 6 + APS MP 7 + APS MP 9 ; or$$

$$APS MP 6 + APS MP 7 + APS MP 10 ; or$$

$$APS MP 6 + APS MP 7 + APS MP 11 ; or$$

$$APS MP 6 + APS MP 8 + APS MP 9 ; or$$

$$APS MP 6 + APS MP 8 + APS MP 10 ; or$$

$$APS MP 6 + APS MP 8 + APS MP 11 ; or$$

$$APS MP 6 + APS MP 9 + APS MP 10 ; or$$

$$APS MP 6 + APS MP 9 + APS MP 11 ; or$$

$$APS MP 6 + APS MP 10 + APS MP 11 ; or$$

$$APS MP 7 + APS MP 8 + APS MP 9 ; or$$

$$APS MP 7 + APS MP 8 + APS MP 10 ; or$$

$$APS MP 7 + APS MP 8 + APS MP 11 ; or$$

$$APS MP 7 + APS MP 9 + APS MP 10 ; or$$

$$APS MP 7 + APS MP 9 + APS MP 11 ; or$$

$$APS MP 7 + APS MP 10 + APS MP 11 ; or$$

$$APS MP 8 + APS MP 9 + APS MP 10 ; or$$

$$APS MP 8 + APS MP 9 + APS MP 11 ; or$$

$$APS MP 8 + APS MP 10 + APS MP 11 ; or$$

$$APS MP 9 + APS MP 10 + APS MP 11$$

The present disclosure provides compositions comprising the following bacteriophage combinations (four bacteriophage types per composition):

$$APS MP 1 + APS MP 2 + APS MP 3 + APS MP 4 ; or$$

$$APS MP 1 + APS MP 2 + APS MP 3 + APS MP 5 ; or$$

$$APS MP 1 + APS MP 2 + APS MP 3 + APS MP 6 ; or$$

$$APS MP 1 + APS MP 2 + APS MP 3 + APS MP 7 ; or$$

$$APS MP 1 + APS MP 2 + APS MP 3 + APS MP 8 ; or$$

$$APS MP 1 + APS MP 2 + APS MP 3 + APS MP 9 ; or$$

$$APS MP 1 + APS MP 2 + APS MP 3 + APS MP 10 ; or$$

$$APS MP 1 + APS MP 2 + APS MP 3 + APS MP 11 ; or$$

$$APS MP 1 + APS MP 2 + APS MP 4 + APS MP 5 ; or$$

$$APS MP 1 + APS MP 2 + APS MP 4 + APS MP 6 ; or$$

$$APS MP 1 + APS MP 2 + APS MP 4 + APS MP 7 ; or$$

$$APS MP 1 + APS MP 2 + APS MP 4 + APS MP 8 ; or$$

$$APS MP 1 + APS MP 2 + APS MP 4 + APS MP 9 ; or$$

$$APS MP 1 + APS MP 2 + APS MP 4 + APS MP 10 ; or$$

$$APS MP 1 + APS MP 2 + APS MP 4 + APS MP 11 ; or$$

$$APS MP 1 + APS MP 2 + APS MP 5 + APS MP 6 ; or$$

$$APS MP 1 + APS MP 2 + APS MP 5 + APS MP 7 ; or$$

$$APS MP 1 + APS MP 2 + APS MP 5 + APS MP 8 ; or$$

$$APS MP 1 + APS MP 2 + APS MP 5 + APS MP 9 ; or$$

$$APS MP 1 + APS MP 2 + APS MP 5 + APS MP 10 ; or$$

$$APS MP 1 + APS MP 2 + APS MP 5 + APS MP 11 ; or$$

$$APS MP 1 + APS MP 3 + APS MP 4 + APS MP 5 ; or$$

$$APS MP 1 + APS MP 3 + APS MP 4 + APS MP 6 ; or$$

$$APS MP 1 + APS MP 3 + APS MP 4 + APS MP 7 ; or$$

$$APS MP 1 + APS MP 3 + APS MP 4 + APS MP 8 ; or$$

$$APS MP 1 + APS MP 3 + APS MP 4 + APS MP 9 ; or$$

$$APS MP 1 + APS MP 3 + APS MP 4 + APS MP 10 ; or$$

$$APS MP 1 + APS MP 3 + APS MP 4 + APS MP 11 ; or$$

$$APS MP 1 + APS MP 3 + APS MP 5 + APS MP 6 ; or$$

$$APS MP 1 + APS MP 3 + APS MP 5 + APS MP 7 ; or$$

$$APS MP 1 + APS MP 3 + APS MP 5 + APS MP 8 ; or$$

$$APS MP 1 + APS MP 3 + APS MP 5 + APS MP 9 ; or$$

$$APS MP 1 + APS MP 3 + APS MP 5 + APS MP 10 ; or$$

$$APS MP 1 + APS MP 3 + APS MP 5 + APS MP 11 ; or$$

$$APS MP 1 + APS MP 4 + APS MP 5 + APS MP 6 ; or$$

$$APS MP 1 + APS MP 4 + APS MP 5 + APS MP 7 ; or$$

$$APS MP 1 + APS MP 4 + APS MP 5 + APS MP 8 ; or$$

$$APS MP 1 + APS MP 4 + APS MP 5 + APS MP 9 ; or$$

$$APS MP 1 + APS MP 4 + APS MP 5 + APS MP 10 ; or$$

$$APS MP 1 + APS MP 4 + APS MP 5 + APS MP 11 ; or$$

$$APS MP 2 + APS MP 3 + APS MP 4 + APS MP 5 ; or$$

$$APS MP 2 + APS MP 3 + APS MP 4 + APS MP 6 ; or$$

$$APS MP 2 + APS MP 3 + APS MP 4 + APS MP 7 ; or$$

$$APS MP 2 + APS MP 3 + APS MP 4 + APS MP 8 ; or$$

$$APS MP 2 + APS MP 3 + APS MP 4 + APS MP 9 ; or$$

$$APS MP 2 + APS MP 3 + APS MP 4 + APS MP 10 ; or$$

$$APS MP 2 + APS MP 3 + APS MP 4 + APS MP 11 ; or$$

$$APS MP 2 + APS MP 3 + APS MP 5 + APS MP 6 ; or$$

$$APS MP 2 + APS MP 3 + APS MP 5 + APS MP 6 ; or$$

$$APS MP 2 + APS MP 3 + APS MP 5 + APS MP 7 ; or$$

$$APS MP 2 + APS MP 3 + APS MP 5 + APS MP 8 ; or$$

$$APS MP 2 + APS MP 3 + APS MP 5 + APS MP 9 ; or$$

$$APS MP 2 + APS MP 3 + APS MP 5 + APS MP 10 ; or$$

$$APS MP 2 + APS MP 3 + APS MP 5 + APS MP 11 ; or$$

$$APS MP 2 + APS MP 4 + APS MP 5 + APS MP 6 ; or$$

$$APS MP 2 + APS MP 4 + APS MP 5 + APS MP 7 ; or$$

$$APS MP 2 + APS MP 4 + APS MP 5 + APS MP 8 ; or$$

$$APS MP 2 + APS MP 4 + APS MP 5 + APS MP 9 ; or$$

$$APS MP 2 + APS MP 4 + APS MP 5 + APS MP 10 ; or$$

$$APS MP 2 + APS MP 4 + APS MP 5 + APS MP 11 ; or$$

$$APS MP 3 + APS MP 4 + APS MP 5 + APS MP 6 ; or$$

$$APS MP 3 + APS MP 4 + APS MP 5 + APS MP 7 ; or$$

$$APS MP 3 + APS MP 4 + APS MP 5 + APS MP 8 ; or$$

$$APS MP 3 + APS MP 4 + APS MP 5 + APS MP 9 ; or$$

$$APS MP 3 + APS MP 4 + APS MP 5 + APS MP 10 ; or$$

$$APS MP 3 + APS MP 4 + APS MP 5 + APS MP 11 ; or$$

$$APS MP 4 + APS MP 5 + APS MP 6 + APS MP 7 ; or$$

$$APS MP 4 + APS MP 5 + APS MP 6 + APS MP 8 ; or$$

$$APS MP 4 + APS MP 5 + APS MP 6 + APS MP 9 ; or$$

$$APS MP 4 + APS MP 5 + APS MP 6 + APS MP 10 ; or$$

$$APS MP 4 + APS MP 5 + APS MP 6 + APS MP 11 ; or$$

$$APS MP 5 + APS MP 6 + APS MP 7 + APS MP 8 ; or$$

$$APS MP 5 + APS MP 6 + APS MP 7 + APS MP 9 ; or$$

$$APS MP 5 + APS MP 6 + APS MP 7 + APS MP 10 ; or$$

$$APS MP 5 + APS MP 6 + APS MP 7 + APS MP 11 ; or$$

$$APS MP 6 + APS MP 7 + APS MP 8 + APS MP 9 ; or$$

$$APS MP 6 + APS MP 7 + APS MP 8 + APS MP 10 ; or$$

$$APS MP 6 + APS MP 7 + APS MP 8 + APS MP 11 ; or$$

$$APS MP 7 + APS MP 8 + APS MP 9 + APS MP 10 ; or$$

$$APS MP 7 + APS MP 8 + APS MP 9 + APS MP 11 ; or$$

$$APS MP 8 + APS MP 9 + APS MP 10 + APS MP 11$$

The present disclosure provides compositions comprising the following bacteriophage combinations (five bacteriophage types per composition):

$$APS MP 1 + APS MP 2 + APS MP 3 + APS MP 4 + APS MP 5 ; or$$

$$APS MP 1 + APS MP 2 + APS MP 3 + APS MP 4 + APS MP 6 ; or$$

$$APS MP 1 + APS MP 2 + APS MP 3 + APS MP 4 + APS MP 7 ; or$$

$$APS MP 1 + APS MP 2 + APS MP 3 + APS MP 4 + APS MP 8 ; or$$

$$APS MP 1 + APS MP 2 + APS MP 3 + APS MP 4 + APS MP 9 ; or$$

$$APS MP 1 + APS MP 2 + APS MP 3 + APS MP 4 + APS MP 10 ; or$$

$$APS MP 1 + APS MP 2 + APS MP 3 + APS MP 4 + APS MP 11 ; or$$

$$APS MP 1 + APS MP 2 + APS MP 3 + APS MP 5 + APS MP 6 ; or$$

$$APS MP 1 + APS MP 2 + APS MP 3 + APS MP 5 + APS MP 7 ; or$$

$$APS MP 1 + APS MP 2 + APS MP 3 + APS MP 5 + APS MP 8 ; or$$

$$APS MP 1 + APS MP 2 + APS MP 3 + APS MP 5 + APS MP 9 ; or$$

$$APS MP 1 + APS MP 2 + APS MP 3 + APS MP 5 + APS MP 10 ; or$$

$$APS MP 1 + APS MP 2 + APS MP 3 + APS MP 5 + APS MP 11 or$$

$$APS MP 1 + APS MP 2 + APS MP 4 + APS MP 5 + APS MP 6 or$$

$$APS MP 1 + APS MP 2 + APS MP 4 + APS MP 5 + APS MP 7 or$$

$$APS MP 1 + APS MP 2 + APS MP 4 + APS MP 5 + APS MP 8 or$$

$$APS MP 1 + APS MP 2 + APS MP 4 + APS MP 5 + APS MP 9 or$$

$$APS MP 1 + APS MP 2 + APS MP 4 + APS MP 5 + APS MP 10 or$$

$$APS MP 1 + APS MP 2 + APS MP 4 + APS MP 5 + APS MP 11 or$$

$$APS MP 1 + APS MP 3 + APS MP 4 + APS MP 5 + APS MP 6 ; or$$

$$APS MP 1 + APS MP 3 + APS MP 4 + APS MP 5 + APS MP 7 or$$

$$APS MP 1 + APS MP 3 + APS MP 4 + APS MP 5 + APS MP 8 or$$

$$APS MP 1 + APS MP 3 + APS MP 4 + APS MP 5 + APS MP 9 or$$

$$APS MP 1 + APS MP 3 + APS MP 4 + APS MP 5 + APS MP 10 or$$

$$APS MP 1 + APS MP 3 + APS MP 4 + APS MP 5 + APS MP 11 or$$

$$APS MP 2 + APS MP 3 + APS MP 4 + APS MP 5 + APS MP 6$$

$$APS MP 2 + APS MP 3 + APS MP 4 + APS MP 5 + APS MP 7 or$$

$$APS MP 2 + APS MP 3 + APS MP 4 + APS MP 5 + APS MP 8 or$$

$$APS MP 2 + APS MP 3 + APS MP 4 + APS MP 5 + APS MP 9 or$$

$$APS MP 2 + APS MP 3 + APS MP 4 + APS MP 5 + APS MP 10 or$$

$$APS MP 2 + APS MP 3 + APS MP 4 + APS MP 5 + APS MP 11 or$$

$$APS MP 2 + APS MP 4 + APS MP 5 + APS MP 6 + APS MP 7 or$$

$$APS MP 2 + APS MP 4 + APS MP 5 + APS MP 6 + APS MP 8 or$$

$$APS MP 2 + APS MP 4 + APS MP 5 + APS MP 6 + APS MP 9 or$$

$$APS MP 2 + APS MP 4 + APS MP 5 + APS MP 6 + APS MP 10 or$$

$$APS MP 2 + APS MP 4 + APS MP 5 + APS MP 6 + APS MP 11 or$$

$$APS MP 2 + APS MP 5 + APS MP 6 + APS MP 7 + APS MP 8 or$$

$$APS MP 2 + APS MP 5 + APS MP 6 + APS MP 7 + APS MP 9 or$$

$$APS MP 2 + APS MP 5 + APS MP 6 + APS MP 7 + APS MP 10 or$$

$$APS MP 2 + APS MP 5 + APS MP 6 + APS MP 7 + APS MP 11 or$$

$$APS MP 2 + APS MP 6 + APS MP 7 + APS MP 8 + APS MP 9 or$$

$$APS MP 2 + APS MP 6 + APS MP 7 + APS MP 8 + APS MP 10 or$$

$$APS MP 2 + APS MP 6 + APS MP 7 + APS MP 8 + APS MP 11 or$$

$$APS MP 2 + APS MP 7 + APS MP 8 + APS MP 9 + APS MP 10 or$$

$$APS MP 2 + APS MP 7 + APS MP 8 + APS MP 9 + APS MP 11 or$$

$$APS MP 2 + APS MP 8 + APS MP 9 + APS MP 10 + APS MP 11 or$$

$$APS MP 3 + APS MP 4 + APS MP 5 + APS MP 6 + APS MP 7 ; or$$

$$APS MP 3 + APS MP 4 + APS MP 5 + APS MP 6 + APS MP 8 or$$

$$APS MP 3 + APS MP 4 + APS MP 5 + APS MP 6 + APS MP 9 or$$

$$APS MP 3 + APS MP 4 + APS MP 5 + APS MP 6 + APS MP 10 or$$

$$APS MP 3 + APS MP 4 + APS MP 5 + APS MP 6 + APS MP 11 or$$

$$APS MP 3 + APS MP 5 + APS MP 6 + APS MP 7 + APS MP 7 ; or$$

$$APS MP 3 + APS MP 5 + APS MP 6 + APS MP 7 + APS MP 9 or$$

$$APS MP 3 + APS MP 5 + APS MP 6 + APS MP 7 + APS MP 10 or$$

$$APS MP 3 + APS MP 5 + APS MP 6 + APS MP 7 + APS MP 11 or$$

$$APS MP 3 + APS MP 6 + APS MP 7 + APS MP 8 + APS MP 9 or$$

$$APS MP 3 + APS MP 6 + APS MP 7 + APS MP 8 + APS MP 10 or$$

$$APS MP 3 + APS MP 6 + APS MP 7 + APS MP 8 + APS MP 11 or$$

$$APS MP 3 + APS MP 7 + APS MP 8 + APS MP 9 + APS MP 10 or$$

$$APS MP 3 + APS MP 7 + APS MP 8 + APS MP 9 + APS MP 11 or$$

$$APS MP 3 + APS MP 8 + APS MP 9 + APS MP 10 + APS MP 11 or$$

$$APS MP 4 + APS MP 5 + APS MP 6 + APS MP 7 + APS MP 8 or$$

$$APS MP 4 + APS MP 5 + APS MP 6 + APS MP 7 + APS MP 9 or$$

$$APS MP 4 + APS MP 5 + APS MP 6 + APS MP 7 + APS MP 10 or$$

$$APS MP 4 + APS MP 5 + APS MP 6 + APS MP 7 + APS MP 11 or$$

$$APS MP 4 + APS MP 6 + APS MP 7 + APS MP 8 + APS MP 9 or$$

$$APS MP 4 + APS MP 6 + APS MP 7 + APS MP 8 + APS MP 10 or$$

$$APS MP 4 + APS MP 6 + APS MP 7 + APS MP 8 + APS MP 11 or$$

$$APS MP 4 + APS MP 7 + APS MP 8 + APS MP 9 + APS MP 10 or$$

$$APS MP 4 + APS MP 7 + APS MP 8 + APS MP 9 + APS MP 11 or$$

$$APS MP 4 + APS MP 8 + APS MP 9 + APS MP 10 + APS MP 11 or$$

$$APS MP 5 + APS MP 6 + APS MP 7 + APS MP 8 + APS MP 9 or$$

$$APS MP 5 + APS MP 6 + APS MP 7 + APS MP 8 + APS MP 10 or$$

$$APS MP 5 + APS MP 6 + APS MP 7 + APS MP 8 + APS MP 11 or$$

$$APS MP 5 + APS MP 7 + APS MP 8 + APS MP 9 + APS MP 10 or$$

$$APS MP 5 + APS MP 7 + APS MP 8 + APS MP9 + APS MP 11 or$$

$$APS MP 5 + APS MP 8 + APS MP 9 + APS MP 10 + APS MP 11 or$$

$$APS MP 6 + APS MP 7 + APS MP 8 + APS MP 9 + APS MP 10 or$$

$$APS MP 6 + APS MP 7 + APS MP 8 + APS MP 9 + APS MP 11 or$$

$$APS MP 6 + APS MP 8 + APS MP 9 + APS MP 10 + APS MP 11 or$$

$$APS MP 7 + APS MP 8 + APS MP 9 + APS MP 10 + APS MP 11$$

The present disclosure provides compositions comprising the following bacteriophage combination (six bacteriophage types per composition):

$$APS MP 1 + APS MP 2 + APS MP 3 + APS MP 4 + APS MP 5 + APS MP 6 ; or$$

$$APS MP 1 + APS MP 2 + APS MP 3 + APS MP 4 + APS MP 5 + APS MP 7 ; or$$

$$APS MP 1 + APS MP 2 + APS MP 3 + APS MP 4 + APS MP 5 + APS MP 8 ; or$$

$$APS MP 1 + APS MP 2 + APS MP 3 + APS MP 4 + APS MP 5 + APS MP 9 ; or$$

$$APS MP 1 + APS MP 2 + APS MP 3 + APS MP 4 + APS MP 5 + APS MP 10 ; or$$

$$APS MP 1 + APS MP 2 + APS MP 3 + APS MP 4 + APS MP 5 + APS MP 11 ; or$$

$$APS MP 1 + APS MP 3 + APS MP 4 + APS MP 5 + APS MP 6 + APS MP 7 ; or$$

$$APS MP 1 + APS MP 3 + APS MP 4 + APS MP 5 + APS MP 6 + APS MP 8 ; or$$

$$APS MP 1 + APS MP 3 + APS MP 4 + APS MP 5 + APS MP 6 + APS MP 9 ; or$$

$$APS MP 1 + APS MP 3 + APS MP 4 + APS MP 5 + APS MP 6 + APS MP 10 ; or$$

$$APS MP 1 + APS MP 3 + APS MP 4 + APS MP 5 + APS MP 6 + APS MP 11 ; or$$

$$APS MP 1 + APS MP 3 + APS MP 4 + APS MP 5 + APS MP 6 + APS MP 7 ; or$$

$$APS MP 1 + APS MP 3 + APS MP 4 + APS MP 5 + APS MP 6 + APS MP 8 ; or$$

$$APS MP 1 + APS MP 3 + APS MP 4 + APS MP 5 + APS MP 6 + APS MP 9 ; or$$

$$APS MP 1 + APS MP 3 + APS MP 4 + APS MP 5 + APS MP 6 + APS MP 10 ; or$$

$$APS MP 1 + APS MP 3 + APS MP 4 + APS MP 5 + APS MP 6 + APS MP 11 ; or$$

$$APS MP 1 + APS MP 4 + APS MP 5 + APS MP 6 + APS MP 7 + APS MP 8 ; or$$

$$APS MP 1 + APS MP 4 + APS MP 5 + APS MP 6 + APS MP 7 + APS MP 9 ; or$$

$$APS MP 1 + APS MP 4 + APS MP 5 + APS MP 6 + APS MP 7 + APS MP 10 ; or$$

$$APS MP 1 + APS MP 4 + APS MP 5 + APS MP 6 + APS MP 7 + APS MP 11 ; or$$

$$APS MP 1 + APS MP 5 + APS MP 6 + APS MP 7 + APS MP 8 + APS MP 9 ; or$$

$$APS MP 1 + APS MP 5 + APS MP 6 + APS MP 7 + APS MP 8 + APS MP 10 ; or$$

$$APS MP 1 + APS MP 5 + APS MP 6 + APS MP 7 + APS MP 8 + APS MP 11 ; or$$

$$APS MP 1 + APS MP 6 + APS MP 7 + APS MP 8 + APS MP 9 + APS MP 10 ; or$$

$$APS MP 1 + APS MP 6 + APS MP 7 + APS MP 8 + APS MP 9 + APS MP 11 ; or$$

$$APS MP 1 + APS MP 7 + APS MP 8 + APS MP 9 + APS MP 10 + APS MP 11 ; or$$

$$APS MP 2 + APS MP 3 + APS MP 4 + APS MP 5 + APS MP 6 + APS MP 7 ; or$$

$$APS MP 2 + APS MP 3 + APS MP 4 + APS MP 5 + APS MP 6 + APS MP 8 ; or$$

$$APS MP 2 + APS MP 3 + APS MP 4 + APS MP 5 + APS MP 6 + APS MP 9 ; or$$

$$APS MP 2 + APS MP 3 + APS MP 4 + APS MP 5 + APS MP 6 + APS MP 10 ; or$$

$$APS MP 2 + APS MP 3 + APS MP 4 + APS MP 5 + APS MP 6 + APS MP 11 ; or$$

$$APS MP 2 + APS MP 4 + APS MP 5 + APS MP 6 + APS MP 7 + APS MP 8 ; or$$

$$APS MP 2 + APS MP 4 + APS MP 5 + APS MP 6 + APS MP 7 + APS MP 9 ; or$$

$$APS MP 2 + APS MP 4 + APS MP 5 + APS MP 6 + APS MP 7 + APS MP 10 ; or$$

$$APS MP 2 + APS MP 4 + APS MP 5 + APS MP 6 + APS MP 7 + APS MP 11 ; or$$

$$APS MP 2 + APS MP 5 + APS MP 6 + APS MP 7 + APS MP 8 + APS MP 9 ; or$$

$$APS MP 2 + APS MP 5 + APS MP 6 + APS MP 7 + APS MP 8 + APS MP 10 ; or$$

$$APS MP 2 + APS MP 5 + APS MP 6 + APS MP 7 + APS MP 8 + APS MP 11 ; or$$

$$APS MP 2 + APS MP 6 + APS MP 7 + APS MP 8 + APS MP 9 + APS MP 10 ; or$$

$$APS MP 2 + APS MP 6 + APS MP 7 + APS MP 8 + APS MP 9 + APS MP 11 ; or$$

$$APS MP 2 + APS MP 7 + APS MP 8 + APS MP 9 + APS MP 10 + APS MP 11 ; or$$

$$APS MP 3 + APS MP 4 + APS MP 5 + APS MP 6 + APS MP 7 + APS MP 8 ; or$$

$$APS MP 3 + APS MP 4 + APS MP 5 + APS MP 6 + APS MP 7 + APS MP 9 ; or$$

$$APS MP 3 + APS MP 4 + APS MP 5 + APS MP 6 + APS MP 7 + APS MP 10 ; or$$

$$APS MP 3 + APS MP 4 + APS MP 5 + APS MP 6 + APS MP 7 + APS MP 11 ; or$$

$$APS MP 3 + APS MP 5 + APS MP 6 + APS MP 7 + APS MP 8 + APS MP 9 ; or$$

$$APS MP 3 + APS MP 5 + APS MP 6 + APS MP 7 + APS MP 8 + APS MP 10 ; or$$

$$APS MP 3 + APS MP 5 + APS MP 6 + APS MP 7 + APS MP 8 + APS MP 11 ; or$$

$$APS MP 3 + APS MP 6 + APS MP 7 + APS MP 8 + APS MP 9 + APS MP 10 ; or$$

$$APS MP 3 + APS MP 6 + APS MP 7 + APS MP 8 + APS MP 9 + APS MP 11 ; or$$

$$APS MP 3 + APS MP 7 + APS MP 8 + APS MP 9 + APS MP 10 + APS MP 11 ; or$$

$$APS MP 4 + APS MP 5 + APS MP 6 + APS MP 7 + APS MP 8 + APS MP 9 ; or$$

$$APS MP 4 + APS MP 5 + APS MP 6 + APS MP 7 + APS MP 8 + APS MP 10 ; or$$

$$APS MP 4 + APS MP 5 + APS MP 6 + APS MP 7 + APS MP 8 + APS MP 11 ; or$$

$$APS MP 4 + APS MP 6 + APS MP 7 + APS MP 8 + APS MP 9 + APS MP 10 ; or$$

$$APS MP 4 + APS MP 6 + APS MP 7 + APS MP 8 + APS MP 9 + APS MP 11 ; or$$

$$APS MP 4 + APS MP 7 + APS MP 8 + APS MP 9 + APS MP 10 + APS MP 11 ; or$$

$$APS MP 5 + APS MP 6 + APS MP 7 + APS MP 8 + APS MP 9 + APS MP 10 ; or$$

$$APS MP 5 + APS MP 6 + APS MP 7 + APS MP 8 + APS MP 9 + APS MP 11 ; or$$

$$APS MP 5 + APS MP 7 + APS MP 8 + APS MP 9 + APS MP 10 + APS MP 11 ; or$$

$$APS MP 6 + APS MP 7 + APS MP 8 + APS MP 9 + APS MP 10 + APS MP 11 ; or$$

$$APS MP 6 + APS MP 8 + APS MP 9 + APS MP 10 + APS MP 11 + APS MP 11 ;$$

The present disclosure provides compositions comprising the following bacteriophage combination (seven) bacteriophage types per composition):

$$APS MP 1 + APS MP 2 + APS MP 3 + APS MP 4 + APS MP 5 + APS MP 6 + APS MP 7 ; or$$

$$APS MP 1 + APS MP 2 + APS MP 3 + APS MP 4 + APS MP 5 + APS MP 6 + APS MP 8 ; or$$

$$APS MP 1 + APS MP 2 + APS MP 3 + APS MP 4 + APS MP 5 + APS MP 6 + APS MP 9 ; or$$

$$APS MP 1 + APS MP 2 + APS MP 3 + APS MP 4 + APS MP 5 + APS MP 6 + APS MP 10 ; or$$

$$APS MP 1 + APS MP 2 + APS MP 3 + APS MP 4 + APS MP 5 + APS MP 6 + APS MP 11 ; or$$

$$APS MP 2 + APS MP 3 + APS MP 4 + APS MP 5 + APS MP 6 + APS MP 7 + APS MP 8 ; or$$

$$APS MP 2 + APS MP 3 + APS MP 4 + APS MP 5 + APS MP 6 + APS MP 7 + APS MP 9 ; or$$

$$APS MP 2 + APS MP 3 + APS MP 4 + APS MP 5 + APS MP 6 + APS MP 7 + APS MP 10 ; or$$

$$APS MP 2 + APS MP 3 + APS MP 4 + APS MP 5 + APS MP 6 + APS MP 7 + APS MP 11 ; or$$

$$APS MP 3 + APS MP 4 + APS MP 5 + APS MP 6 + APS MP 7 + APS MP 8 + APS MP 9 ; or$$

$$APS MP 3 + APS MP 4 + APS MP 5 + APS MP 6 + APS MP 7 + APS MP 8 + APS MP 10 ; or$$

$$APS MP 3 + APS MP 4 + APS MP 5 + APS MP 6 + APS MP 7 + APS MP 8 + APS MP 11 ; or$$

$$APS MP 4 + APS MP 5 + APS MP 6 + APS MP 7 + APS MP 8 + APS MP 9 + APS MP 10 ; or$$

$$APS MP 4 + APS MP 5 + APS MP 6 + APS MP 7 + APS MP 8 + APS MP 9 + APS MP 11 ; or$$

$$APS MP 5 + APS MP 6 + APS MP 7 + APS MP 8 + APS MP 9 + APS MP 10 + APS MP 11 ;$$

The present disclosure provides compositions comprising the following bacteriophage combination (eight bacteriophage types per composition):

$$APS MP 1 + APS MP 2 + APS MP 3 + APS MP 4 + APS MP 5 + APS MP 6 + APS MP 7 + APS MP 8 ; or$$

$$APS MP 1 + APS MP 2 + APS MP 3 + APS MP 4 + APS MP 5 + APS MP 6 + APS MP 7 + APS MP 9 ; or$$

$$APS MP 1 + APS MP 2 + APS MP 3 + APS MP 4 + APS MP 5 + APS MP 6 + APS MP 7 + APS MP 10 ; or$$

$$APS MP 1 + APS MP 2 + APS MP 3 + APS MP 4 + APS MP 5 + APS MP 6 + APS MP 7 + APS MP 11 ; or$$

$$APS MP 2 + APS MP 3 + APS MP 4 + APS MP 5 + APS MP 6 + APS MP 7 + APS MP 8 + APS MP 9 ; or$$

$$APS MP 2 + APS MP 3 + APS MP 4 + APS MP 5 + APS MP 6 + APS MP 7 + APS MP 8 + APS MP 10 ; or$$

$$APS MP 2 + APS MP 3 + APS MP 4 + APS MP 5 + APS MP 6 + APS MP 7 + APS MP 8 + APS MP 11 ; or$$

$$APS MP 3 + APS MP 4 + APS MP 5 + APS MP 6 + APS MP 7 + APS MP 8 + APS MP 9 + APS MP 10 ; or$$

$$APS MP 3 + APS MP 4 + APS MP 5 + APS MP 6 + APS MP 7 + APS MP 8 + APS MP 9 + APS MP 11 ; or$$

$$APS MP 4 + APS MP 5 + APS MP 6 + APS MP 7 + APS MP 8 + APS MP 9 + APS MP 10 + APS MP 11 ; or$$

The present disclosure provides compositions comprising the following bacteriophage combination (nine) bacteriophage types per composition):

$$APS MP 1 + APS MP 2 + APS MP 3 + APS MP 4 + APS MP 5 + APS MP 6 + APS MP 7 + APS MP 8 + APS MP 9$$

$$APS MP 1 + APS MP 2 + APS MP 3 + APS MP 4 + APS MP 5 + APS MP 6 + APS MP 7 + APS MP 8 + APS MP 10 ; or$$

$$APS MP 1 + APS MP 2 + APS MP 3 + APS MP 4 + APS MP 5 + APS MP 6 + APS MP 7 + APS MP 8 + APS MP 11 ; or$$

$$APS MP 1 + APS MP 2 + APS MP 3 + APS MP 4 + APS MP 5 + APS MP 6 + APS MP 7 + APS MP 8 + APS MP 9 ; or$$

$$APS MP 2 + APS MP 3 + APS MP 4 + APS MP 5 + APS MP 6 + APS MP 7 + APS MP 8 + APS MP 9 + APS MP 10 ; or$$

$$APS MP 2 + APS MP 3 + APS MP 4 + APS MP 5 + APS MP 6 + APS MP 7 + APS MP 8 + APS MP 9 + APS MP 11 ; or$$

$$APS MP 2 + APS MP 4 + APS MP 5 + APS MP 6 + APS MP 7 + APS MP 8 + APS MP 9 + APS MP 10 + APS MP 11 ; or$$

$$APS MP 3 + APS MP 4 + APS MP 5 + APS MP 6 + APS MP 7 + APS MP 8 + APS MP 9 + APS MP 10 + APS MP 11$$

The present disclosure provides compositions comprising the following bacteriophage combination (ten) bacteriophage types per composition):

$$APS MP 1 + APS MP 2 + APS MP 3 + APS MP 4 + APS MP 5 + APS MP 6 + APS MP 7 + APS MP 8 + APS MP 9 + APS MP 10 ; or$$

$$APS MP 1 + APS MP 2 + APS MP 3 + APS MP 4 + APS MP 5 + APS MP 6 + APS MP 7 + APS MP 8 + APS MP 9 + APS MP 11 ; or$$

$$APS MP 1 + APS MP 3 + APS MP 4 + APS MP 5 + APS MP 6 + APS MP 7 + APS MP 8 + APS MP 9 + APS MP 10 ; or$$

$$APS MP 2 + APS MP 3 + APS MP 4 + APS MP 5 + APS MP 6 + APS MP 7 + APS MP 8 + APS MP 9 + APS MP 10 + APS MP 11.$$

The present disclosure provides compositions comprising the following bacteriophage combination (eleven) bacteriophage types per composition):

$$APS MP 1 + APS MP 2 + APS MP 3 + APS MP 4 + APS MP 5 + APS MP 6 + APS MP 7 + APS MP 8 + APS MP 9 + APS MP 10 + APS MP 11.$$

Without wishing to be bound by theory, the inventors have discovered that compositions comprising one or more of the phage disclosed herein are particularly useful as the bacteria to which they are specific do not mutate sufficiently over time to render the composition useless and/or there are likely multiple attachment sites for the bacteriophage on the bacterial surface. A composition of this disclosure may further comprise an excipient, diluent or buffer. For example, a composition may comprise an amount of a formulated “phage buffer”. The phage for use (one or more of those described herein) may be suspended in a volume of a suitable phage buffer. A useful phage buffer may comprise a buffer and one or more salts (for example inorganic salts). For example, a useful phage buffer may comprise a volume of Tris, an amount of NaCl and MgSO4.

A suitable formulation may be:
50 mM Tris (pH 7.5);
100 mM NaCl; and
8 mM MgSo4.

Without wishing to be bound by theory, certain combinations of phage (bacteriophage) may be particularly useful and/or exhibit a synergistic effect. That is to say, that the decontaminating, lysogenic or lysis effect of a combination of different phage types is greater than the sum of the decontaminating effect of the individual phage types. For example, compositions comprising:
(i) APS MP1, APS MP2, APS MP3 and APS MP4;
(ii) APS MP1, APS MP2, APS MP3, APS MP4 and APS MP6;
(iii) APS MP9;

$$APS MP 9 + APS MP 1 ; \qquad ( iv )$$

$$APS MP 9 + APS MP 2 ; \qquad ( v )$$

$$APS MP 9 + APS MP 3 ; \qquad ( vi )$$

$$APS MP 9 + APS MP 4 ; \qquad ( vii )$$

$$APS MP 9 + APS MP 7 ; \qquad ( viii )$$

$$APS MP 9 + APS MP 8 ; \qquad ( ix )$$

$$APS MP 9 + APS MP 10 ; \qquad ( x )$$

$$APS MP 9 + APS MP 11 ; \qquad ( xi )$$

$$APS MP 9 + APS MP 1 \_ + APS MP 2 ; \qquad ( xii )$$

$$APS MP 9 + APS MP 1 + APS MP 4 ; \qquad ( xiii )$$

$$APS MP 9 + APS MP 1 + APS MP 10 ; \qquad ( xiv )$$

$$APS MP 9 + APS MP 1 + APS MP 11 ; \qquad ( xv )$$

$$APS MP 9 + APS MP 1 + APS MP 2 + APS MP 4 ; \qquad ( xvi )$$

$$APS MP 9 + APS MP 1 + APS MP 2 + APS MP 4 + APS MP 10 ; or \qquad ( xvii )$$

$$APS MP 9 + APS MP 1 + APS MP 2 + APS MP 4 + APS MP 10 + APS MP 11 ; \qquad ( xviii )$$

may provide exemplary compositions.

By way of example, a composition of this disclosure may be effective against about (for example approximately 1%, 5%, 10%, 15%, 20%, 25%, 30%, 35%, 40%, 45%, 50%, 55% or 60% of the bacterial species/strains known to cause mushroom blotch. In the context of the efficacy of a composition of this invention, the term “about” may mean±/−2%, 3%, 4% or 5%.

The compositions of this invention may be formulated for application to mushrooms and/or mushroom growing substrates.

The compositions may be provided as liquid formulations which may be poured, sprayed, dropped or wiped onto a mushroom or a cultivation substrate (a compost or casing) therefor. For example, a composition may comprise one or more of the phage (bacteriophage) described herein suspended and/or dissolved in a liquid solvent. Optionally, the solution and/or suspension may comprise one or more of a stabiliser, a buffer, an excipient and/or the like. Advantageously, a liquid formulation may be applied by a user onto a mushroom or a cultivation substrate (a compost or casing) therefor directly without the need to generate add any additional water and/or solvent to the composition.

A composition may be provided as dry, desiccated and/or lyophilised formulation. For example, a composition and/or formulation may be prepared (and provided) as a freeze-dried composition. A composition and/or formulation may be spray-dried. Advantageously, a dry formulation may facilitate transport, may be stable for longer periods of time, and/or may require less packaging and/or space for storage.

The number of bacteriophage present in a composition of this invention may be dictated by the likely or predicted number, diversity and/or type of bacteria to be decontaminated. The more diverse the bacteria flora (comprising for example different species or types of blotch-causing bacteria) the more phage may be required to ensure a suitable level of decontamination is achieved. A composition or formulation of this invention may comprise any suitable concentration or amount of phage. For example the total phage content may be 10² pfu/ml, 10³ pfu/ml, 10⁴ pfu/ml, 10⁵ pfu/ml, 10⁶ pfu/ml, 10⁷ pfu/ml, 10⁸ pfu/ml, 10⁹ pfu/ml 10¹⁰ pfu/ml, 10¹¹ pfu/ml, 10¹² pfu/ml, 10¹³ pfu/ml, 10¹⁴ pfu/ml or 10¹⁵ pfu/ml. For example, suitable concentrations may be between about 10⁴ pfu/ml and about 10⁸ pfu/ml. For example, a formulation may contain about 10⁶ pfu/ml or about 10⁷ pfu/ml. The formulation or composition may comprise a suitable quantity, concentration or amount (for example a quantity commensurate with the PFU amounts detailed above) of one or more different phage. The (PFU) quantity of each phage may be same or different.

The compositions of this invention may be used during mushroom growing, processing and/or storage procedures, optionally as a supplement to standard washing, cleaning and/or decontamination techniques. For example, a growing or harvested mushroom crop may be watered, washed or cleaned in a composition of this invention. The composition may be used concurrently or together (or before or after) any routine irrigation or watering procedure that is part of the standard mushroom growing/farming protocol.

Additionally, or alternatively, prior to storing, packaging or bagging a harvested mushroom crop, a composition of this invention (which composition may comprise one or more of the disclosed bacteriophage) may be applied (by dropping, spraying or the like) to the mushroom crop.

In a further aspect, the present invention provides a method of preparing mushrooms for storage or packaging, method comprising contacting mushrooms to be stored or packed with one or more of the phage and/or compositions described herein.

In a yet further aspect, the invention provides stored or packed mushroom treated with a composition of this invention.

### DETAILED DESCRIPTION

The present invention will now be described in detail with reference to the following Figures which show:

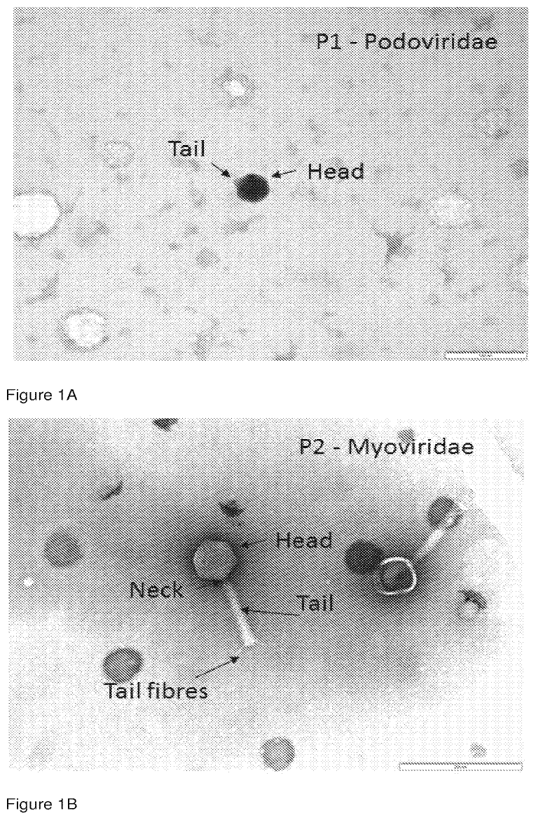

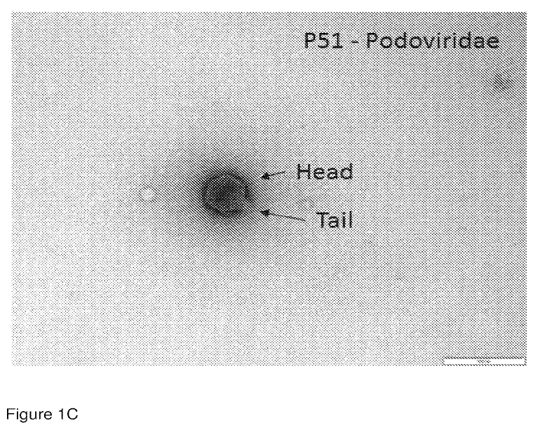

FIGS. 1A, B and C: bacteriophage (1A: Podoviridae; 1B Myoviridae and 1C Podoviridae) analysis by transmission electron microscopy.

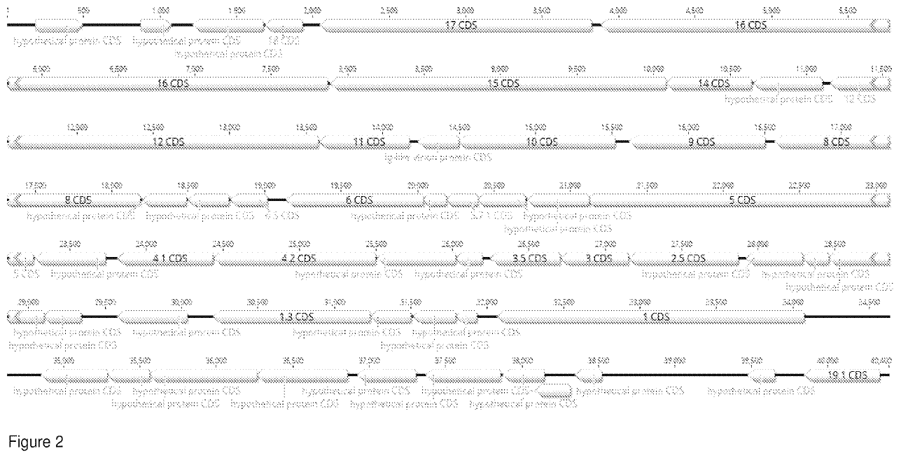

FIG. 2: Genome detail of P1

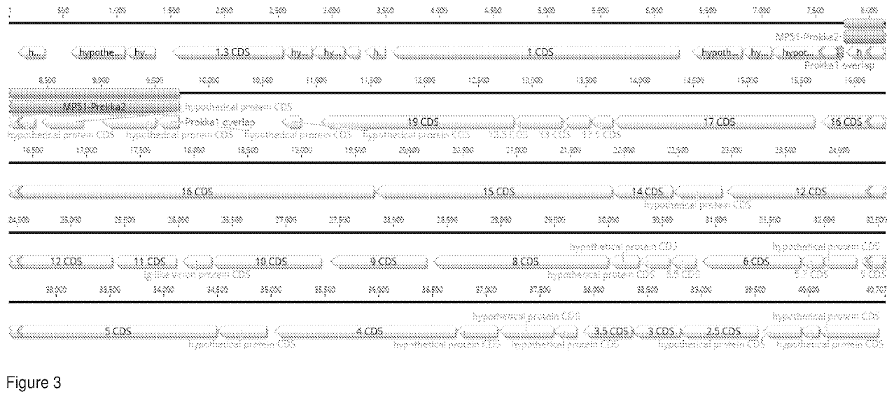

FIG. 3 Genome detail of P51

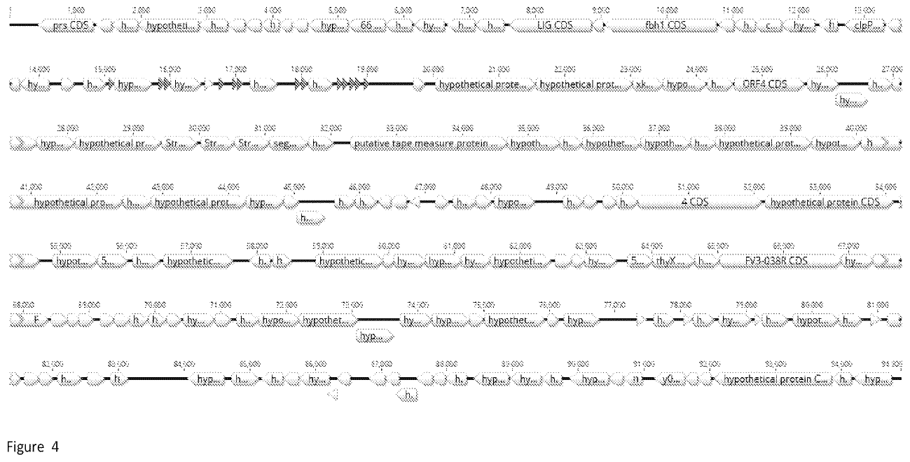

FIG. 4: Genome detail of P2

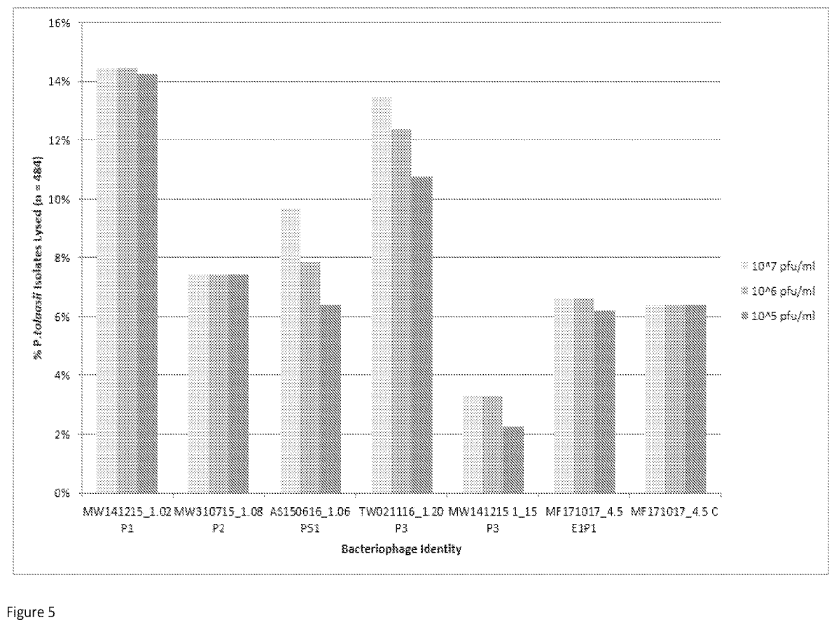

FIG. 5: Individual Phage: % P. tolaasii isolates lysed (n=484)

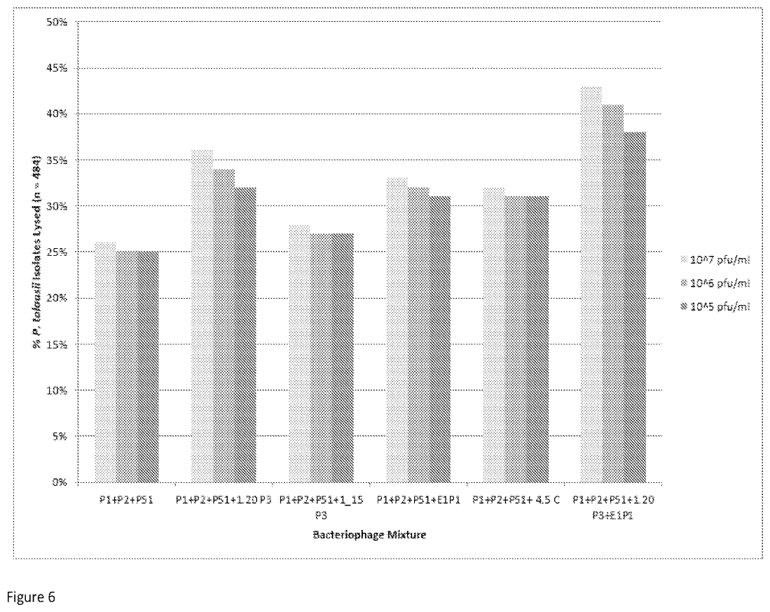

FIG. 6: Combined Phage: % P. tolaasii isolates lysed (n=484)

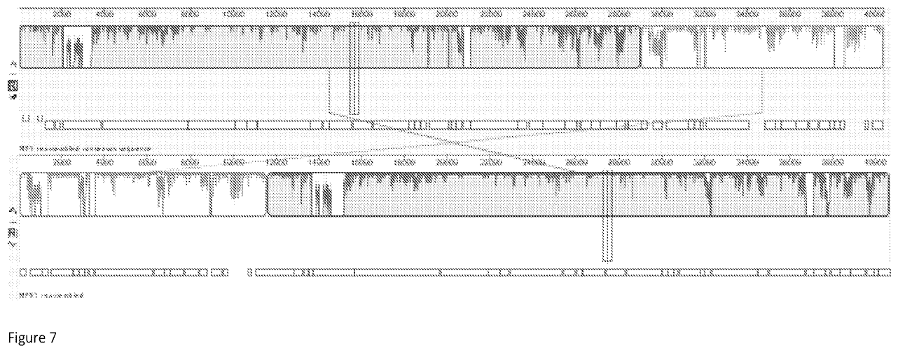

FIG. 7: Comparison between P1 and P51 genome sequences

### EXAMPLE 1

### Genome and Morphology Analysis of Mushroom Phage P1, P2 and P51

The suspensions of each bacteriophage were analysed by transmission electron microscopy (TEM): P1 and P51 were classified as Podoviridae due to the presence of a very short tail and P2 was identified as a member of the Myoviridae due to the presence of a contractile tail and a clearly visible neck.

The capsid sizes of all phages and the tail length of P2 were measured from the TEM images (FIGS. 1A, B and C).

TABLE 2
Capsid sizes and tail length of bacteriophage

| | | | Mean head | Mean head | Mean tail |
|---|---|---|---|---|---|
| | | | diameter | volume | length |
| Bacteriophage | Tail | Classification | (nm) | (nm³) | (nm) |
| P1 | Absent | Podoviridae | 43 | 2.25 × 10⁴ | — |
| P2 | Contractile | Myoviridae | 70 | 1.09 × 10⁵ | 119 |
| P51 | Absent | Podoviridae | 48 | 3.51 × 10⁴ | — |

### Genome Sequence Analysis

The phage genomic DNA was purified from pure phage suspension by phenol-chloroform method and submitted to BaseClear BV, The Netherlands, for full genome sequencing, assembly and annotation.

### P1 and P51 (See FIG. 7)

The complete genome sizes of both phage are about 40.4 KB in size. The phage share overall 84.8% sequence identity. The average sequence identity of the identified ORFs is above 92%, with the notable exception of 17CDS in each of the genomes annotated as Tail Fibre Protein, which share 23% DNA sequence identity and 38.4% amino acid sequence identity and 50.9% sequence similarity. This sequence is responsible for a big mismatch of the aligned sequences and suggest significant differences in the affinity of P1 and P51 to cell surface receptors.

The genome of P1 (top) shows rearrangement of the end block of the sequence (highlighted in green) in comparison to the genome sequence of P51 (bottom). The colour boxes highlight the blocks of each sequence sharing homology. The height of the peaks within each box represent sequence identity within the blocks. The area with the lowest sequence identity corresponds to the coding sequence of the tail fibre protein 17CDS.

Analysis of the genome sequences was performed by BaseClear using Prokka v1.12. Overall there were 51 ORFs and 50 ORFs, respectively recognised in P1 and P51, out of which products of 24 ORFs in each genome had a function predictable based on SwissProt analysis:

TABLE 3
ORFs with predicted function in the genome of P1

| ORF name | annotation ID | Predicted function |
|---|---|---|
| 1.3 CDS | Prokka_00004 | DNA ligase |
| 1 CDS | Prokka_00009 | DNA-directed RNA polymerase |
| 19 CDS | Prokka_00014 | Terminase, large subunit gp19 |
| 18.5 CDS | Prokka_00015 | Spanin, inner membrane subunit |
| 18 CDS | Prokka_00016 | DNA maturase A |
| 17.5 CDS | Prokka_00017 | Holin |
| 17 CDS | Prokka_00018 | Tail fiber protein |
| 16 CDS | Prokka_00019 | Peptidoglycan hydrolase gp16 |
| 15 CDS | Prokka_00020 | Internal virion protein gp15 |
| 14 CDS | Prokka_00021 | Internal virion protein gp14 |
| 12 CDS | Prokka_00023 | Tail tubular protein gp12 |
| 11 CDS | Prokka_00024 | Tail tubular protein gp11 |
| Ig-like virion | Prokka_00025 | Ig-like virion protein |
| protein CDS | | |
| 10 CDS | Prokka_00026 | Major capsid protein 10A |
| 9 CDS | Prokka_00027 | Capsid assembly scaffolding protein |
| 8 CDS | Prokka_00028 | Portal protein |
| 6.5 CDS | Prokka_00031 | putative gene 6.5 protein |
| 6 CDS | Prokka_00032 | Exonuclease |
| 5.7 CDS | Prokka_00033 | Gene 5.7 protein |
| 5 CDS | Prokka_00035 | DNA-directed DNA polymerase |
| 4 CDS | Prokka_00037 | DNA primase/helicase |
| 3.5 CDS | Prokka_00041 | Endolysin |
| 3 CDS | Prokka_00042 | Endonuclease I |
| 2.5 CDS | Prokka_00043 | Single-stranded DNA-binding protein |
| | | gp2.5 |

TABLE 4
ORFs with predicted function in the genome of P51

| ORF name | annotation ID | Predicted function |
|---|---|---|
| 1.3 CDS | Prokka_00004 | DNA ligase |
| 1 CDS | Prokka_00009 | DNA-directed RNA polymerase |
| 19 CDS | Prokka_00014 | Terminase, large subunit gp19 |
| 18.5 CDS | Prokka_00015 | Spanin, inner membrane subunit |
| 18 CDS | Prokka_00016 | DNA maturase A |
| 17.5 CDS | Prokka_00017 | Holin |
| 17 CDS | Prokka_00018 | Tail fiber protein |
| 16 CDS | Prokka_00019 | Peptidoglycan hydrolase gp16 |
| 15 CDS | Prokka_00020 | Internal virion protein gp15 |
| 14 CDS | Prokka_00021 | Internal virion protein gp14 |
| 12 CDS | Prokka_00023 | Tail tubular protein gp12 |
| 11 CDS | Prokka_00024 | Tail tubular protein gp11 |
| Ig-like virion | Prokka_00025 | Ig-like virion protein |
| protein CDS | | |
| 10 CDS | Prokka_00026 | Major capsid protein 10A |
| 9 CDS | Prokka_00027 | Capsid assembly scaffolding protein |
| 8 CDS | Prokka_00028 | Portal protein |
| 6.5 CDS | Prokka_00031 | putative gene 6.5 protein |
| 6 CDS | Prokka_00032 | Exonuclease |
| 5.7 CDS | Prokka_00033 | Gene 5.7 protein |
| 5 CDS | Prokka_00035 | DNA-directed DNA polymerase |
| 4 CDS | Prokka_00037 | DNA primase/helicase |
| 3.5 CDS | Prokka_00041 | Endolysin |
| 3 CDS | Prokka_00042 | Endonuclease I |
| 2.5 CDS | Prokka_00043 | Single-stranded DNA-binding protein |
| | | gp2.5 |

### P2

The complete genome sequence of P2 is 94.9 KB and does not align with the other two sequences, which is to be expected from an unrelated bacteriophage. A total of 177 ORF was recognised by Prokka out of which 35 had function predictable by SwissProt.

TABLE 5
ORFs with predicted function in the genome of P2

| ORF name | annotation ID | Predicted function |
|---|---|---|
| prs CDS | Prokka_00001 | Ribose-phosphate pyrophosphokinase |
| 66 CDS | Prokka_00012 | Gene 66 protein |
| hegA CDS | Prokka_00013 | H-N-H endonuclease F-TfIIV |
| LIG CDS | Prokka_00017 | DNA ligase |
| fbh1 CDS | Prokka_00019 | F-box DNA helicase protein 1 |
| comEB CDS | Prokka_00022 | ComE operon protein 2 |
| cIpP CDS | Prokka_00025 | 3.4.21.92 cIpP ATP-dependent Clp |
| | | protease proteolytic subunit |
| tRNA-GIn | Prokka_00030 | tRNA-Gln |
| tRNA-Trp | Prokka_00032 | tRNA-Trp |
| tRNA-Arg | Prokka_00033 | tRNA-Arg |
| tRNA-Asp | Prokka_00036 | tRNA-Asp |
| tRNA-Thr | Prokka_00037 | tRNA-Thr |
| tRNA-Ile | Prokka_00038 | tRNA-Ile |
| tRNA-Asn | Prokka_00040 | tRNA-Asn |
| tRNA-Pro | Prokka_00043 | tRNA-Pro |
| tRNA-Ala | Prokka_00044 | tRNA-Ala |
| tRNA-His | Prokka_00045 | tRNA-His |
| tRNA-Leu | Prokka_00046 | tRNA-Leu |
| tRNA-Gly | Prokka_00047 | tRNA-Gly |
| xkdF CDS | Prokka_00051 | Phage-like element PBSX protein |
| | | XkdF |
| ORF4 CDS | Prokka_00054 | Major capsid protein |
| Structural | Prokka_00061 | Structural protein ORF10 |
| protein | | |
| ORF10 CDS | | |
| Structural | Prokka_00062 | Structural protein ORF10 |
| protein | | |
| ORF10 CDS | | |
| Structural | Prokka_00063 | Structural protein ORF10 |
| protein | | |
| ORF10 CDS | | |
| segD CDS | Prokka_00064 | Putative endonuclease segD |
| putative tape | Prokka_00066 | putative tape measure protein |
| measure protein | | |
| CDS | | |
| 4 CDS | Prokka_00093 | DNA primase/helicase |
| 59 1 CDS | Prokka_00095 | Gene 59 protein |
| 59 2 CDS | Prokka_00097 | Gene 59 protein |
| 59 3 CDS | Prokka_00111 | Gene 59 protein |
| thyX CDS | Prokka_00112 | Flavin-dependent thymidylate |
| | | synthase |
| FV3-038R CDS | Prokka_00114 | Putative ribonucleoside-diphosphate |
| | | reductase large subunit |
| FV3-067L CDS | Prokka_00116 | Putative ribonucleoside-diphosphate |
| | | reductase small subunit 067L |
| nhaP CDS | Prokka_00171 | Na(+)/H(+) antiporter NhaP |
| y06D CDS | Prokka_00172 | putative 17.5 kDa protein in tk-vs |
| | | intergenic region |

12 of the recognised ORFs were identified as tRNA loci.

TABLE 6
Percentage of P. tolaasii isolates lysed
with individual phage compositions

| | 10\hat{ }7 | 10\hat{ }6 | 10\hat{ }5 |
|---|---|---|---|
| | pfu/ml | pfu/ml | pfu/ml |
| MW141215_1.02 | 14% | 14% | 14% |
| P1 | | | |
| MW310715_1.08 | 7% | 7% | 7% |
| P2 | | | |
| AS150616_1.06 | 10% | 8% | 6% |
| P51 | | | |
| TW021116_1.20 | 13% | 12% | 11% |
| P3 | | | |
| MW141215 1_15 | 3% | 3% | 2% |
| P3 | | | |
| MF171017_4.5 | 7% | 7% | 6% |
| E1P1 | | | |
| MF171017_4.5 C | 6% | 6% | 6% |

TABLE 7
Percentage of P. tolaasii isolates
lysed with combined phage compositions

| | 10\hat{ }7 | 10\hat{ }6 | 10\hat{ }5 |
|---|---|---|---|
| | pfu/ml | pfu/ml | pfu/ml |
| P1 + P2 + P51 | 26% | 25% | 25% |
| P1 + P2 + P51 + 1.20 P3 | 36% | 34% | 32% |
| P1 + P2 + P51 + 1_15 P3 | 28% | 27% | 27% |
| P1 + P2 + P51 + E1P1 | 33% | 32% | 31% |
| P1 + P2 + P51 + 4.5 C | 32% | 31% | 31% |
| P1 + P2 + P51 + 1.20 | 43% | 41% | 38% |
| P3 + E1P1 | | | |

### EXAMPLE 2

TABLE 8
Percentage of P. tolaasii isolates lysed
with individual phage compositions

| | 10\hat{ }7 | 10\hat{ }6 | 10\hat{ }5 |
|---|---|---|---|
| | pfu/ml | pfu/ml | pfu/ml |
| MP1 | 12% | 11% | 11% |
| MP2 | 9 | 8 | 8 |
| MP3 | 9% | 7% | 6% |
| MP4 | 11% | 9% | 8% |
| MP5 | 4% | 4% | 3% |
| MP6 | 8% | 8% | 7% |
| MP7 | 9% | 8% | 8% |
| MP8 | 7% | 6% | 6% |
| MP9 | 22% | 22% | 21% |
| MP10 | 11% | 10% | 10% |
| MP11 | 6% | 5% | 4% |

TABLE 9
Percentage of P. tolaasii isolates
lysed with combined phage compositions

| | 10\hat{ }7 | 10\hat{ }6 | 10\hat{ }5 |
|---|---|---|---|
| | pfu/ml | pfu/ml | pfu/ml |
| MP9 | 22% | 20% | 19% |
| MP9 + MP1 | 34% | 32% | 31% |
| MP9 + MP2 | 27% | 25% | 24% |
| MP9 + MP3 | 30% | 27% | 25% |
| MP9 + MP4 | 33% | 30% | 28% |
| MP9 + MP7 | 30% | 27% | 26% |
| MP9 + MP8 | 28% | 26% | 25% |
| MP9 + MP10 | 30% | 28% | 27% |
| MP9 + MP11 | 27% | 25% | 24% |
| MP9 + MP1 + MP2 | 39% | 37% | 36% |
| MP9 + MP1 + MP4 | 42% | 39% | 37% |
| MP9 + MP1 + MP10 | 42% | 40% | 39% |
| MP9 + MP1 + MP11 | 40% | 36% | 35% |
| MP9 + MP1 + MP2 + MP4 | 47% | 44% | 42% |
| MP9 + MP1 + MP2 + MP4 + MP10 | 56% | 52% | 50% |
| MP9 + MP1 + MP2 + MP4 + | 60% | 57% | 54% |
| MP10 + MP11 | | | |

What is claimed is:

1. A method of decontaminating food or a food growing medium, substrate or compost of one or more microorganisms, said method comprising contacting the food and/or the food growing medium, substrate or compost with one or more phages.

2. The method of claim 1, wherein the food is selected from the group consisting of:
(i) any food, food stuff and/or food product for human or animal consumption;
(ii) manufactured, harvested, farmed or processed food, food stuff, produce or product;
(iii) crops;
(iv) cereals;
(v) dairy produce;
(vi) meat;
(vii) vegetables and root vegetables;
(viii) edible leaves, herbs or salad;
(ix) fruit;
(x) seeds;
(xi) tubers;
(xii) fungi and/or mushrooms;
(xiii) bagged salad; and
(ix) potatoes.

3. The method of claim 1, wherein the food is mushrooms.

4. The method of claim 1, wherein the food growing medium, substrate or compost is a medium, substrate or compost for the growing or cultivation of mushrooms.

5. The method of claim 1, wherein the one or more microorganisms are part of the microbial flora of the food and/or food growing medium, substrate or compost.

6. The method of claim 1, wherein the one or more microorganisms are a microbial contaminant or pathogen of the food and/or food growing medium, substrate or compost.

7. The method of claim 1, wherein the one or more microorganisms are associated with spoilage, premature ripening and/or rotting of the food.

8. The method, of claim 1, wherein the method comprises the phage deposited as NCIMB 42864.

9. The method of claim 1, wherein the one or more phages are selected from the group consisting of:
(i) Phage deposited as NCIMB 42864;
(ii) Phage deposited as NCIMB 42866;
(iii) Phage deposited as NCIMB 42868;
(iv) Phage deposited as NCIMB 43014;
(v) Phage deposited as NCIMB 43016;
(vi) Phage deposited as NCIMB 43018;
(vii) Phage deposited as NCIMB 43467;
(viii) Phage deposited as NCIMB 43469;
(ix) Phage deposited as NCIMB 43471;
(x) Phage deposited as NCIMB 43473; and
(xi) Phage deposited as NCIMB 43475.

10. The method of claim 1, wherein the one or more phages comprise, consist essentially of or consist of:
(i) Phage deposited as NCIMB 42864;
(ii) Phage deposited as NCIMB 42866;
(iii) Phage deposited as NCIMB 42868;
(iv) Phage deposited as NCIMB 43014;
(v) Phage deposited as NCIMB 43018;
(vii) Phage deposited as NCIMB 43467;
(viii) Phage deposited as NCIMB 43469;
(ix) Phage deposited as NCIMB 43471;
(x) Phage deposited as NCIMB 43473; and
(xi) Phage deposited as NCIMB 43475.

11. Food and/or stored or packed food treated with the method of claim 1.

12. The composition of claim 25, wherein the excipient, the diluent and/or the buffer comprises tris, NaCl and MgSO4.

13. The composition of claim 25, wherein the excipient, the diluent and/or the buffer comprises:
(i) 50 mM Tris (pH 7.5);
(ii) 100 mM NaCl; and
(iii) 8 mM MgSo4.

14. A method of treating or preventing mushroom blotch, said method comprising contacting mushrooms at risk of mushroom blotch and/or a medium or substrate for growing or cultivating mushroom with one or more phages selected from the group consisting of:
(i) Phage deposited as NCIMB 42864;
(ii) Phage deposited as NCIMB 42866;
(iii) Phage deposited as NCIMB 42868;
(iv) Phage deposited as NCIMB 43014;
(v) Phage deposited as NCIMB 43016;
(vi) Phage deposited as NCIMB 43018;
(vii) Phage deposited as NCIMB 43467;
(viii) Phage deposited as NCIMB 43469;
(ix) Phage deposited as NCIMB 43471;
(x) Phage deposited as NCIMB 43473; and
(xi) Phage deposited as NCIMB 43475.

15. A composition for use in treating or preventing mushroom blotch, said composition comprising one or more phages selected from the group consisting of:
(i) Phage deposited as NCIMB 42864;
(ii) Phage deposited as NCIMB 42866;
(iii) Phage deposited as NCIMB 42868;
(iv) Phage deposited as NCIMB 43014;
(v) Phage deposited as NCIMB 43016;
(vi) Phage deposited as NCIMB 43018;
(vii) Phage deposited as NCIMB 43467;
(viii) Phage deposited as NCIMB 43469;
(ix) Phage deposited as NCIMB 43471;
(x) Phage deposited as NCIMB 43473; and
(xi) Phage deposited as NCIMB 43475.

16. The method of claim 1, wherein the one ore more microorganisms cause or contribute to mushroom blotch and/or Pseudomonas tolaasii.

17. A composition for the decontamination of food and/or a food growing medium, substrate or compost of one or more microorganisms, said composition comprising one or more phages.

18. The composition of claim 17, wherein the food is mushrooms.

19. The composition of claim 17, wherein the food growing medium, substrate or compost is a medium, substrate or compost for the growing or cultivation of mushrooms.

20. The composition of claim 17, wherein the one or more microorganisms are part of or a pathogen/contaminant of the microbial flora of the food and/or food growing medium, substrate or compost.

21. The composition of claim 17, wherein the one or more microorganisms cause or contribute to mushroom blotch and/or Pseudomonas tolaasii.

22. The composition of claim 17, wherein the composition comprises the phage deposited as NCIMB 42864.

23. The composition of claim 17, wherein the one or more phages are selected from the group consisting of:
(i) Phage deposited as NCIMB 42864;
(ii) Phage deposited as NCIMB 42866;
(iii) Phage deposited as NCIMB 42868;
(iv) Phage deposited as NCIMB 43014;
(v) Phage deposited as NCIMB 43016;
(vi) Phage deposited as NCIMB 43018;
(vii) Phage deposited as NCIMB 43467;
(viii) Phage deposited as NCIMB 43469;
(ix) Phage deposited as NCIMB 43471;
(x) Phage deposited as NCIMB 43473; and
(xi) Phage deposited as NCIMB 43475.

24. The composition of claim 17, wherein the one or more phages comprise, consist essentially of or consist of:
(i) Phage deposited as NCIMB 42864;
(ii) Phage deposited as NCIMB 42866;
(iii) Phage deposited as NCIMB 42868;
(iv) Phage deposited as NCIMB 43014;
(v) Phage deposited as NCIMB 43018;
(vii) Phage deposited as NCIMB 43467;
(viii) Phage deposited as NCIMB 43469;
(ix) Phage deposited as NCIMB 43471;
(x) Phage deposited as NCIMB 43473; and
(xi) Phage deposited as NCIMB 43475.

25. The composition of claim 17, wherein the composition comprises an excipient, diluent and/or buffer.
